(19) United States
(12) Patent Application Publication
(10) Pub. No.: US 2024/0076394 A1
(43) Pub. Date: Mar. 7, 2024

(54) MODULATING THE IMMUNE RESPONSE USING ANTI-CD30 ANTIBODY-DRUG CONJUGATES

(71) Applicant: Seagen Inc., Bothell, WA (US)

(72) Inventors: Ryan A. HEISER, Lake Stevens, WA (US); Bryan M. GROGAN, Snohomish, WA (US)

(21) Appl. No.: 18/039,473

(22) PCT Filed: Dec. 2, 2021
(86) PCT No.: PCT/US2021/061660

§ 371 Date: May 30, 2023

Related U.S. Application Data
(60) Provisional application No. 63/121,044, filed on Dec. 3, 2020.

(51) Int. Cl. C07K 16/28; A61P 35/04

(52) U.S. Cl. CPC C07K 16/2875; A61P 35/04; A61K 2039/545; C07K 2317/565

(57) ABSTRACT

Provided herein are anti-CD30 antibody-drug conjugates and methods of using the same to modulate the immune response in a subject.

### CROSS-REFERENCE TO RELATED APPLICATIONS

This application claims priority to and benefit of U.S. Provisional Application No. 63/121,044, filed Dec. 3, 2020, the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682005840SEQLIST.TXT, date recorded: Dec. 1, 2021, size: 5,833 bytes).

### TECHNICAL FIELD

The present invention relates to anti-CD30 antibody-drug conjugates and methods of using the same to modulate the immune response in a subject.

### BACKGROUND

CD30 is a 120 kilodalton membrane glycoprotein (Froese et al., 1987, J. Immunol. 139: 2081-87) and a member of the TNF-receptor superfamily that has been shown to be a marker of malignant cells in Hodgkin's lymphoma and anaplastic large cell lymphoma (ALCL), a subset of non-Hodgkin's lymphoma (NHL) (Dürkop et al., 1992, Cell 88:421-427). CD30 has been found to be highly expressed on the cell surface of all Hodgkin's lymphomas and the majority of ALCL (Josimovic-Alasevic et al., 1989, Eur. J. Immunol. 19:157-162).

CD30 was originally identified by the monoclonal antibody Ki-1 (Schwab et al., 1982, Nature 299:65-67). This monoclonal antibody was developed against Hodgkin and Reed-Sternberg (H-RS) cells, the malignant cells of Hodgkin's lymphoma. A second monoclonal antibody, capable of binding a formalin resistant epitope different from that recognized by Ki-1, was subsequently described (Schwarting et al., 1989, Blood 74:1678-1689). The identification of four additional antibodies resulted in the creation of the CD30 cluster at the Third Leucocyte Typing Workshop in 1986 (McMichael, A., ed., 1987, Leukocyte Typing III (Oxford: Oxford University Press)). Monoclonal antibodies specific for the CD30 antigen have been explored as vehicles for the delivery of cytostatic drugs, plant toxins and radioisotopes to cancerous cells expressing CD30 in both preclinical models and clinical studies (Engert et al., 1990, Cancer Research 50:84-88; Barth et al., 2000, Blood 95:3909-3914). In patients with Hodgkin's lymphoma, targeting of the CD30 antigen could be achieved with low doses of the anti-CD30 antibody, BerH2 (Falini et al., 1992, British Journal of Haematology 82:38-45). Yet, despite successful in vivo targeting of the malignant tumor cells, none of the patients experienced tumor regression. In a subsequent clinical trial, the toxin saporin was chemically conjugated to the BerH2 antibody and all four patients demonstrated rapid and substantial reductions in tumor mass (Falini et al., 1992, Lancet 339:1195-1196). However, in vitro studies using an antibody-drug conjugate (ADC) where the toxin dgA was conjugated to the Ki-1 antibody demonstrated only moderate efficacy when administered to patients with resistant HL in a Phase 1 clinical trial (Schnell et al., 2002, Clinical Cancer Research, 8(6):1779-1786).

Brentuximab vedotin (BV) is a CD30-directed antibody-drug conjugate (ADC) consisting of 3 components: 1) the chimeric IgG1 antibody cAC10, specific for human CD30; 2) the microtubule-disrupting agent monomethyl auristatin E (IMAE); and 3) a protease-cleavable linker that covalently attaches MMAE to cAC10. Targeted delivery of MMAE to CD30-expressing tumor cells is the primary mechanism of action of brentuximab vedotin. Binding of MMAE to tubulin disrupts the microtubule network within the cell, subsequently inducing cell cycle arrest and apoptotic death of the cell. Other nonclinical studies suggest additional contributory mechanisms of action, including antibody-dependent cellular phagocytosis; bystander effects on nearby cells in the tumor microenvironment due to released MMAE; and immunogenic cell death due to endoplasmic reticulum stress, which drives exposure of immune activating molecules that can promote a T-cell response.

In addition to ADCs comprising antibodies conjugated to MMAE, there is another class of ADCs that is sufficiently active, while having a suitable toxicity profile, to warrant clinical development. This class includes the camptothecin conjugates (i.e., camptothecin-containing ADCs), such as SGN-CD30C. Camptothecin has a different mechanism of action compared to NMMAE, namely inhibiting topoisomerase I rather than disrupting microtubules. Unlike MMAE, camptothecin-based therapies do not cause peripheral neuropathy clinically.

T regulatory cells (Tregs) are essential modulators of T cell immune responses, limiting chronic inflammation and protecting normal tissues from autoimmunity. T regulatory cells are also implicated in maintaining immune-suppressive conditions in the tumor microenvironment, abrogating cytotoxic anti-tumor immunosurveillance. Analysis of clinical tumor samples has shown increased densities of intratumoral Tregs associated with poor clinical outcomes in a number of cancer types (Fridman, 2012, Nature Reviews Cancer; Charoentong, 2017, Cell Reports 18: 248-262). Recent transcriptomic analyses of intratumoral Tregs isolated from breast, lung, and colorectal cancer tissues showed TNFSFR8 (CD30) to be among transcripts differentially upregulated compared to Tregs isolated from adjacent normal tissue and circulating in blood (Plitas, 2016, Immunity, 45: 1122-1134; De Simone, 2016, Immunity, 45: 1135-1147). The functional significance of heightened CD30 transcript expression in Tregs remains unclear. Given the protective role of Tregs in promoting immune homeostasis in normal tissues, there is considerable interest in developing cancer therapeutics that preferentially target intratumoral Tregs, while sparing those in non-diseased tissues. Therefore, there appears to be a need for therapies that can selectively control the activity of immune cells that are involved in pathogenesis of cancer, such as the activity of T regulatory cells.

All references cited herein, including patent applications, patent publications, and scientific literature, are herein incorporated by reference in their entirety, as if each individual reference were specifically and individually indicated to be incorporated by reference.

### SUMMARY

Provided herein is a method of decreasing the number of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the number of CD30⁺ Treg cells is decreased relative to the number of CD30⁺ Treg cells in the subject prior to administration of the antibody-drug conjugate. In some embodiments, the decrease in the activity of CD30 Treg cells is relative to the activity of CD30⁺ Treg cells in the subject prior to administration of the antibody-drug conjugate. In some embodiments, the CD30+ Treg cells are inducible T regulatory (iTreg) cells. In some embodiments, the CD30+ Treg cells are peripheral blood T regulatory (pbTreg) cells. In some embodiments, the anti-CD30 antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6. In some embodiments, the anti-CD30 antibody or antigen-binding fragment thereof of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody or antigen-binding fragment thereof of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8. In some embodiments, the anti-CD30 antibody or antigen-binding fragment thereof of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8. In some embodiments, the anti-CD30 antibody or antigen-binding fragment thereof of the antibody-drug conjugate comprises a heavy chain variable region comprising the amino acid sequence of SEQ ID NO: 7 and a light chain variable region comprising the amino acid sequence of SEQ ID NO: 8. In some embodiments, the anti-CD30 antibody or antigen-binding fragment thereof of the antibody-drug conjugate is cAC10. In some embodiments, the antibody-drug conjugate comprises an anti-CD30 antibody or an antigen-binding fragment thereof conjugated to a camptothecin or a functional analog thereof or a functional derivative thereof forming a camptothecin conjugate of Formula (IC):

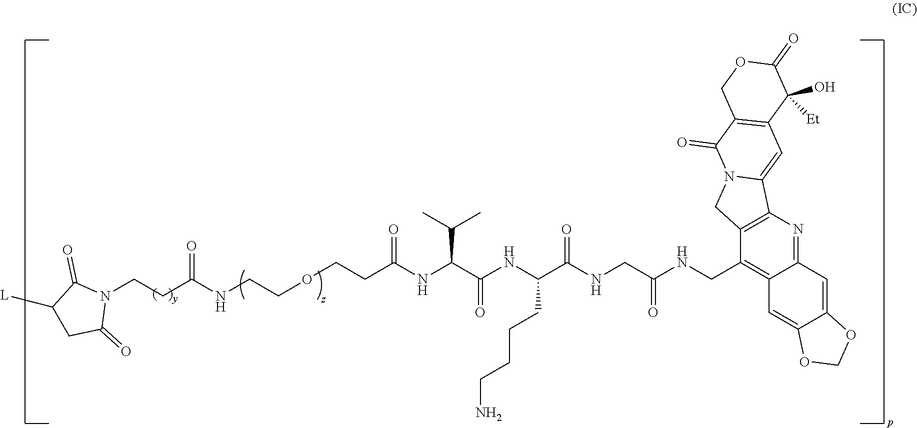

or a pharmaceutically acceptable salt thereof, wherein:
L is the anti-CD30 antibody or an antigen-binding fragment thereof,
y is 1, 2, 3, or 4, or is 1 or 4,
z is an integer from 2 to 12, or is 2, 4, 8, or 12, and
p is 1-16, or is 2, 3, 4, 5, 6, 7, 8, 9, or 10, or is 2, 4 or 8. In some embodiments, y is 1. In some embodiments, z is 8. In some embodiments, p is 8. In some embodiments, the antibody-drug conjugate is SGN-CD30C. In some embodiments, the antibody-drug conjugate is administered at a dose of 0.01 mg/kg to 5 mg/kg of the subject's bodyweight. In some embodiments, the antibody-drug conjugate is administered at a dose of 0.1 mg/kg to 2 mg/kg of the subject's bodyweight. In some embodiments, the antibody-drug conjugate is administered at a dose of about 0.1 mg/kg of the subject's bodyweight. In some embodiments, the antibody-drug conjugate is administered at a dose of about 0.5 mg/kg of the subject's bodyweight. In some embodiments, the antibody-drug conjugate is administered to the subject once about every 3 weeks. In some embodiments, the antibody-drug conjugate is administered to the subject once every 3 weeks. In some embodiments, the antibody-drug conjugate is administered to the subject on about day 1 of about a 21-day treatment cycle. In some embodiments, the antibody-drug conjugate is administered to the subject on day 1 of a 21-day treatment cycle. In some embodiments, the antibody-drug conjugate is administered by intravenous infusion. In some embodiments, the method further comprises the administration of granulocyte-colony stimulating factor (G-CSF) to the subject. In some embodiments, the G-CSF is administered 1 to 3 days after the administration of the antibody-drug conjugate. In some embodiments, the G-CSF is selected from the group consisting of filgrastim, PEG-filgrastim, lenograstim, and tbo-filgrastim. In some embodiments, the method further comprises administering one or more additional therapeutic agents capable of modulating the immune response. In some embodiments, the subject has cancer. In some embodiments, the cancer is a hematologic cancer. In some embodiments, the cancer is selected from the group consisting of Hodgkin lymphoma, non-Hodgkin lymphoma, anaplastic large cell lymphoma, peripheral T-cell lymphoma, or mycosis fungoides. In some embodiments, the cancer is Hodgkin lymphoma. In some embodiments, the Hodgkin lymphoma is classical Hodgkin lymphoma (cHL). In some embodiments, the cancer is non-Hodgkin lymphoma. In some embodiments, the non-Hodgkin lymphoma is diffuse large B-cell lymphoma (DLBCL). In some embodiments, the DLBCL is germinal-center B-cell like (GCB). In some embodiments, the DLBCL is non-GCB. In some embodiments, the cancer is anaplastic large cell lymphoma. In some embodiments, the anaplastic large cell lymphoma is systemic anaplastic large cell lymphoma. In some embodiments, the anaplastic large cell lymphoma is primary cutaneous anaplastic large cell lymphoma. In some embodiments, the non-Hodgkin lymphoma is a mature T-cell lymphoma. In some embodiments, the non-Hodgkin lymphoma is cutaneous T-cell lymphoma (CTCL). In some embodiments, the cancer is peripheral T-cell lymphoma. In some embodiments, the peripheral T-cell lymphoma is angioimmunoblastic T-cell lymphoma. In some embodiments, the cancer is mycosis fungoides. In some embodiments, the cancer is a non-hematologic cancer. In some embodiments, the non-hematologic cancer is a carcinoma. In some embodiments, the non-hematologic cancer is a sarcoma. In some embodiments, the non-hematologic cancer is a solid tumor. In some embodiments, the cancer is a CD30+ cancer, while in other embodiments, the cancer is a CD30− cancer. In some embodiments, the cancer is an advanced stage cancer. In some embodiments, the advanced stage cancer is a stage 3 or stage 4 cancer. In some embodiments, the advanced stage cancer is metastatic cancer. In some embodiments, the subject has been previously treated with one or more therapeutic agents and did not respond to the treatment. In some embodiments, the subject has been previously treated with one or more therapeutic agents and relapsed after the treatment. In some embodiments, the subject has been previously treated with one or more therapeutic agents and has experienced disease progression during treatment. In some embodiments, the subject has previously received allogenic stem cell transplant to treat the cancer. In some embodiments, the subject has previously received autologous stem cell transplant to treat the cancer. In some embodiments, the subject relapsed following stem cell transplant. In some embodiments, the subject has previously received CAR-T therapy. In some embodiments, the cancer is recurrent cancer. In some embodiments, the subject has not previously been treated for the cancer. In some embodiments, the subject has not been previously treated with an antibody-drug conjugate that binds to CD30. In some embodiments, at least 1% of the cancer cells in the subject express CD30. In some embodiments, administering the antibody-drug conjugate to the subject results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the antibody-drug conjugate to the subject. In some embodiments, one or more therapeutic effects in the subject is improved after administration of the antibody-drug conjugate relative to a baseline. In some embodiments, the one or more therapeutic effects is selected from the group consisting of: objective response rate, duration of response, time to response, progression free survival and overall survival. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the duration of response to the conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject is a human.

Also provided herein is a pharmaceutical composition an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, wherein the composition is for use in the method of any of the embodiments herein.

Also provided herein is a kit comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, and instructions for using the kit in the method of any of the embodiments herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1A-1B is a series of graphs showing the ability of SGN-CD30C to deplete CD30+ Tregs in vitro. SGN-CD30C depleted total CD30+ Tregs (FIG. 1A), and peripheral blood CD30+ Tregs (FIG. 1B). CD30+ Treg counts are shown as a percent of control. hIgG-7782 is a non-binding control antibody-drug conjugate.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“CD30” or “TNFRSF8” refers to a receptor that is a member of the tumor necrosis factor receptor superfamily. CD30 is a transmembrane glycoprotein expressed on activated CD4⁺ and CD8⁺ T cells and B cells, and virally-infected lymphocytes. CD30 interacts with TRAF2 and TRAF3 to mediate signal transduction that leads to activation of NF-κB. CD30 acts as a positive regulator of apoptosis, and it has been shown to limit the proliferative potential of auto-reactive CD8 effector T cells. CD30 is also expressed by various forms of lymphoma, including Hodgkin lymphoma (CD30 is expressed by Reed-Sternberg cells) and non-Hodgkin lymphoma (e.g., diffuse large B-cell lymphoma (DLBCL), peripheral T-cell lymphoma (PTCL), and cutaneous T-cell lymphoma (CTCL).

The term “immunotherapy” refers to the treatment of a subject afflicted with, at risk of contracting, or suffering a recurrence of a disease by a method comprising inducing, enhancing, suppressing, or otherwise modifying an immune response.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as CIq, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to CD30 is substantially free of antibodies that bind specifically to antigens other than CD30). An isolated antibody that binds specifically to CD30 can, however, have cross-reactivity to other antigens, such as CD30 molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-CD30 antibody includes a conjugate of an anti-CD30 antibody with a small molecule drug (e.g., a camptothecin or a functional analog thereof or a functional derivative thereof).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-CD30 antibody is an antibody that binds to the antigen CD30.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻⁰ M or less, or about 10⁻¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate, which in the context of the present invention refers to an anti-CD30 antibody, which is coupled to a drug moiety (e.g., a camptothecin) as described in the present application.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

The terms “Treg” or “regulatory T cell” refer to CD4⁺ T cells that suppresses CD4 CD25⁺ and CD8⁺ T cell proliferation and/or effector function, or that otherwise down−modulate an immune response. Notably, Treg may down-regulate immune responses mediated by Natural Killer cells, Natural Killer T cells as well as other immune cells.

The terms “regulatory T cell function” or “a function of Treg” are used interchangeably to refer to any biological function of a Treg that results in a reduction in CD4 CD25⁺ or CD8⁺ T cell proliferation or a reduction in an effector T cell-mediated immune response. Treg function can be measured via techniques established in the art. Non-limiting examples of useful in vitro assays for measuring Treg function include Transwell suppression assays as well as in vitro assays in which the target conventional T cells (Tconv) and Tregs purified from human peripheral blood or umbilical cord blood (or murine spleens or lymph nodes) are optionally activated by anti-CD3⁺ anti-CD28 coated beads (or antigen-presenting cells (APCs) such as, e.g., irradiated splenocytes or purified dendritic cells (DCs) or irradiated PBMCs) followed by in vitro detection of conventional T cell proliferation (e.g., by measuring incorporation of radioactive nucleotides (such as, e.g., [H]-thymidine) or fluorescent nucleotides, or by Cayman Chemical MTT Cell Proliferation Assay Kit, or by monitoring the dilution of a green fiuorochrome ester CFSF or Seminaphtharhodafluor (SNARF-1) dye by flow cytometry). Other common assays measure T cell cytokine responses. Useful in vivo assays of Treg function include assays in animal models of diseases in which Tregs play an important role, including, e.g., (1) homeostasis model (using naïve homeostatically expanding CD4⁺ T cells as target cells that are primarily suppressed by Tregs), (2) inflamnmatory bowel disease (IBD) recovery model (using Thl T cells (Thl7) as target cells that are primarily suppressed by Tregs), (3) experimental autoimmune encephalomyelitis (EAE) model (using Thl 7 and Thl T cells as target cells that are primarily suppressed by Tregs), (4) B16 melanoma model (suppression of antitumor immunity) (using CD8⁺ T cells as target cells that are primarily suppressed by Tregs), (5) suppression of colon inflammation in adoptive transfer colitis where naïve CD4⁺CD45RB^{M} Tconv cells are transferred into RagV mice, and (6) Foxp3 rescue model (using lymphocytes as target cells that are primarily suppressed by Tregs). According to one protocol, all of the models require mice for donor T cell populations as well as Ragl^{-/-} or Foxp3 mice for recipients. For more details on various useful assays see, e.g., Collison and Vignali, In Vitro Treg Suppression Assays, Chapter 2 in Regulatory T Cells: Methods and Protocols, Methods in Molecular Biology, Kassiotis and Liston eds., Springer, 2011, 707:21-37; Workman et al, In Vivo Treg Suppression Assays, Chapter 9 in Regulatory T Cells: Methods and Protocols, Methods in Molecular Biology, Kassiotis and Liston eds., Springer, 2011, 119-156; Takahashi et al, Int. Immunol, 1998, 10: 1969-1980; Thornton et al, J. Exp. Med., 1998, 188:287-296; Collison et al, J. Imnmunol, 2009, 182:6121-6128; Thornton and Shevach, J. Exp. Med., 1998, 188:287-296; Asseman et al, J. Exp. Med., 1999, 190:995-1004; Dieckmann et al, J. Exp. Med., 2001, 193: 1303-1310; Belkaid, Nature Reviews, 2007, 7:875-888; Tang and Bluestone, Nature Immunology, 2008, 9:239-244; Bettini and Vignali, Curr. Opin. Immunol, 2009, 21:612-618; Dannull et al, J Clin Invest, 2005, 115(12):3623-33; Tsaknaridis, et al, J Neurosci Res., 2003, 74:296-308.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 0.3 mg/kg of an anti-CD30 antibody or an anti-CD30 antibody-drug conjugate, one can calculate and use the appropriate amount of the anti-CD30 antibody or anti-CD30 antibody-drug conjugate (i.e., 18 mg) for administration to said subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., an anti-CD30 antibody or an anti-CD30 antibody-drug conjugate). For example, a subject with 60 kg body weight and a subject with 100 kg body weight would receive the same dose (e.g., 18 mg of an anti-CD30 antibody or an anti-CD30 antibody-drug conjugate).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration for the anti-CD30 antibody-drug conjugate include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy (e.g., an anti-CD30 antibody-drug conjugate as described herein) or at the beginning of administration of the therapy. The baseline value can be compared to a reference value in order to determine the reduction or improvement of a symptom of a CD30-associated disease contemplated herein (e.g., cancer). The terms “reference” or “reference value” used interchangeably herein can refer to a measurement or characterization of a symptom after administration of the therapy (e.g., an anti-CD30 antibody-drug conjugate as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-CD30 antibody-drug conjugate is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Invention

In one aspect, the methods disclosed herein are used in place of standard of care therapies. The anti-CD30 antibody-drug conjugates described herein are used to decrease the number of CD30⁺ T regulatory cells and/or decrease the activity of CD30⁺ T regulatory cells in a subject, which can result in improved treatment compared to standard of care therapies. In some embodiments, the subject has cancer. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014, available at: www.nccn.org/professionals/physician_gls/f_guidelines.asp, last accessed May 14, 2014).

One aspect of the invention provides a method of modulating the immune response in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. Another aspect of the invention provides a method of decreasing the number of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the number of CD30⁺ Treg cells is decreased relative to the number of CD30 Treg cells in the subject prior to administration of the antibody-drug conjugate. Another aspect of the invention provides a method of decreasing the activity of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the decrease in the activity of CD30 Treg cells is relative to the activity of CD30 Treg cells in the subject prior to administration of the antibody-drug conjugate. In some embodiments, the CD30+ Treg cells are inducible T regulatory (iTreg) cells. In some embodiments, the CD30+ Treg cells are peripheral blood T regulatory (pbTreg) cells. In some embodiments, the anti-CD30 antibody or antigen-binding fragment thereof of the antibody-drug conjugate comprises the complementary determining regions (CDRs) of brentuximab, or a biosimilar thereof. In some embodiments, the anti-CD30 antibody or antigen-binding fragment thereof of the antibody-drug conjugate comprises the complementary determining regions (CDRs) of brentuximab. In some embodiments, the anti-CD30 antibody or antigen-binding fragment thereof of the antibody-drug conjugate comprises the heavy chain variable region and the light chain variable region of brenutximab, or a biosimilar thereof. In some embodiments, the anti-CD30 antibody or antigen-binding fragment thereof of the antibody-drug conjugate comprises the heavy chain variable region and the light chain variable region of brentuximab. In some embodiments, the anti-CD30 antibody of the antibody-drug conjugate is brentuximab or a biosimilar thereof. In some embodiments, the anti-CD30 antibody of the antibody-drug conjugate is brentuximab. In some embodiments, the antibody-drug conjugate is SGN-CD30C or a biosimilar thereof. In some embodiments, the antibody-drug conjugate is SGN-CD30C. In some embodiments, the subject has cancer. In some embodiments, the cancer is a hematologic cancer. In some embodiments, the cancer is selected from the group consisting of Hodgkin lymphoma, non-Hodgkin lymphoma, anaplastic large cell lymphoma, peripheral T-cell lymphoma, or mycosis fungoides. In some embodiments, the cancer is Hodgkin lymphoma. In some embodiments, the Hodgkin lymphoma is classical Hodgkin lymphoma (cHL). In some embodiments, the cancer is non-Hodgkin lymphoma. In some embodiments, the non-Hodgkin lymphoma is diffuse large B-cell lymphoma (DLBCL). In some embodiments, the DLBCL is germinal-center B-cell like (GCB). In some embodiments, the DLBCL is non-GCB. In some embodiments, the cancer is anaplastic large cell lymphoma. In some embodiments, the anaplastic large cell lymphoma is systemic anaplastic large cell lymphoma. In some embodiments, the anaplastic large cell lymphoma is primary cutaneous anaplastic large cell lymphoma. In some embodiments, the non-Hodgkin lymphoma is a mature T-cell lymphoma. In some embodiments, the non-Hodgkin lymphoma is cutaneous T-cell lymphoma (CTCL). In some embodiments, the cancer is peripheral T-cell lymphoma. In some embodiments, the peripheral T-cell lymphoma is angioimmunoblastic T-cell lymphoma. In some embodiments, the cancer is mycosis fungoides. In some embodiments, the cancer is a non-hematologic cancer. In some embodiments, the non-hematologic cancer is a carcinoma. In some embodiments, the non-hematologic cancer is a sarcoma. In some embodiments, the non-hematologic cancer is a solid tumor. In some embodiments, the cancer is a CD30+ cancer, while in other embodiments, the cancer is a CD30− cancer. In some embodiments, the cancer is an advanced stage cancer. In some embodiments, the advanced stage cancer is a stage 3 or stage 4 cancer. In some embodiments, the advanced stage cancer is metastatic cancer. In some embodiments, the subject has been previously treated with one or more therapeutic agents and did not respond to the treatment. In some embodiments, the subject has been previously treated with one or more therapeutic agents and relapsed after the treatment. In some embodiments, the subject has been previously treated with one or more therapeutic agents and has experienced disease progression during treatment. In some embodiments, the subject has previously received allogenic stem cell transplant to treat the cancer. In some embodiments, the subject has previously received autologous stem cell transplant to treat the cancer. In some embodiments, the subject relapsed following stem cell transplant. In some embodiments, the subject has previously received CAR-T therapy. In some embodiments, the subject relapsed after CAR-T therapy. In some embodiments, the cancer is recurrent cancer. In some embodiments, the subject has not previously been treated for the cancer. In some embodiments, the subject has not been previously treated with an antibody-drug conjugate that binds to CD30. In some embodiments, at least 1% of the cancer cells in the subject express CD30. In some embodiments, the subject is a human.

A. Anti-CD30 Antibodies and Antibody-Drug Conjugates

i. Anti-CD30 Antibody

In one aspect, the therapy of the present disclosure utilizes an anti-CD30 antibody or an antigen-binding fragment thereof. CD30 receptors are members of the tumor necrosis factor receptor superfamily involved in limiting the proliferative potential of autoreactive CD8 effector T cells. Antibodies targeting CD30 can potentially be either agonists or antagonists of these CD30 mediated activities. In some embodiments, the anti-CD30 antibody is conjugated to a therapeutic agent (e.g., an anti-CD30 antibody-drug conjugate).

Murine anti-CD30 mAbs known in the art have been generated by immunization of mice with Hodgkin's disease (HD) cell lines or purified CD30 antigen. AC10, originally termed C10 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16. See also U.S. Pat. No. 7,090,843, incorporated herein by reference.

Generally, anti-CD30 antibodies of the disclosure bind CD30, e.g., human CD30, and exert cytostatic and cytotoxic effects on cells expressing CD30. Anti-CD30 antibodies of the disclosure are preferably monoclonal, and may be multispecific, human, humanized or chimeric antibodies, single chain antibodies, Fab fragments, F(ab′) fragments, fragments produced by a Fab expression library, and CD30 binding fragments of any of the above. In some embodiments, the anti-CD30 antibodies of the disclosure specifically bind CD30. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-CD30 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-CD30 antibodies or antigen-binding fragments thereof are human, murine (e.g., mouse and rat), donkey, sheep, rabbit, goat, guinea pig, camelid, horse, or chicken.

The anti-CD30 antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-CD30 antibodies of the present disclosure may be described or specified in terms of the particular CDRs they comprise. In certain embodiments antibodies of the disclosure comprise one or more CDRs of AC10. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC10, and in which said antibody or derivative thereof immunospecifically binds CD30.

In one aspect, the anti-CD30 antibody is AC10. In some embodiments, the anti-CD30 antibody is cAC10. cAC10 is a chimeric IgG1 monoclonal antibody that specifically binds CD30. cAC10 induces growth arrest of CD30⁺ cell lines in vitro and has pronounced antitumor activity in severe combined immunodeficiency (SCID) mouse xenograft models of Hodgkin disease. See Francisco et al., Blood 102(4):1458-64 (2003). AC10 antibody and cAC10 antibody are described in U.S. Pat. Nos. 9,211,319 and 7,090,843.

In one aspect, anti-CD30 antibodies that compete with AC10 antibody and/or cAC10 antibody binding to CD30 are provided. Anti-CD30 antibodies that bind to the same epitope as AC10 antibody and cAC10 antibody are also provided.

In one aspect, provided herein is an anti-CD30 antibody comprising 1, 2, 3, 4, 5, or 6 of the CDR sequences of the AC10 antibody. In one aspect, provided herein is an anti-CD30 antibody comprising 1, 2, 3, 4, 5, or 6 of the CDR sequences of the cAC10 antibody. In some embodiments, the CDR is a Kabat CDR or a Chothia CDR.

In one aspect, provided herein is an anti-CD30 antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-CD30 antibody described herein may comprise any suitable framework variable domain sequence, provided that the antibody retains the ability to bind CD30 (e.g., human CD30). As used herein, heavy chain framework regions are designated “HC-FR1-FR4,” and light chain framework regions are designated “LC-FR1-FR4.” In some embodiments, the anti-CD30 antibody comprises a heavy chain variable domain framework sequence of SEQ ID NO:9, 10, 11, and 12 (HC-FR1, HC-FR2, HC-FR3, and HC-FR4, respectively). In some embodiments, the anti-CD30 antibody comprises a light chain variable domain framework sequence of SEQ ID NO:13, 14, 15, and 16 (LC-FR1, LC-FR2, LC-FR3, and LC-FR4, respectively).

In one embodiment, an anti-CD30 antibody comprises a heavy chain variable domain comprising a framework sequence and hypervariable regions, wherein the framework sequence comprises the HC-FR1-HC-FR4 amino acid sequences of SEQ ID NO:9 (HC-FR1), SEQ ID NO:10 (HC-FR2), SEQ ID NO:11 (HC-FR3), and SEQ ID NO:12 (HC-FR4), respectively; the CDR-H1 comprises the amino acid sequence of SEQ ID NO:1; the CDR-H2 comprises the amino acid sequence of SEQ ID NO:2; and the CDR-H3 comprises the amino acid sequence of SEQ ID NO:3.

In one embodiment, an anti-CD30 antibody comprises a light chain variable domain comprising a framework sequence and hypervariable regions, wherein the framework sequence comprises the LC-FR1-LC-FR4 amino acid sequences of SEQ ID NO: 13 (LC-FR1), SEQ ID NO:14 (LC-FR2), SEQ ID NO:15 (LC-FR3), and SEQ ID NO:16 (LC-FR4), respectively; the CDR-L1 comprises the amino acid sequence of SEQ ID NO:4; the CDR-L2 comprises the amino acid sequence of SEQ ID NO:5; and the CDR-L3 comprises the amino acid sequence of SEQ ID NO:6.

In some embodiments of the anti-CD30 antibodies described herein, the heavy chain variable domain comprises the amino acid sequence of

(SEQ ID NO: 7)
QIQLQQSGPEVVKPGASVKISCKASGYTFTDYYITWVKQKPGQGLEWIG
WIYPGSGNTKYNEKFKGKATLTVDTSSSTAFMQLSSLTSEDTAVYFCAN
YGNYWFAYWGQGTQVTVSA

and the light chain variable domain comprises the amino acid sequence of

(SEQ ID NO: 8)
DIVLTQSPASLAVSLGQRATISCKASQSVDFDGDSYMNWYQQKPGQPPK
VLIYAASNLESGIPARFSGSGSGTDFTLNIHPVEEEDAATYYCQQSNED
PWTFGGGTKLEIK.

In some embodiments of the anti-CD30 antibodies described herein, the heavy chain CDR sequences comprise the following:

a) CDR-H1
(DYYIT (SEQ ID NO: 1));
b) CDR-H2
(WIYPGSGNTKYNEKFKG (SEQ ID NO: 2));
and
c) CDR-H3
(YGNYWFAY (SEQ ID NO:3)).

In some embodiments of the anti-CD30 antibodies described herein, the heavy chain FR sequences comprise the following:

a) HC-FR1
(QIQLQQSGPEVVKPGASVKISCKASGYTFT (SEQ ID NO: 9));
b) HC-FR2
(WVKQKPGQGLEWIG (SEQ ID NO: 10));
c) HC-FR3
(KATLTVDTSSSTAFMQLSSLTSEDTAVYFCAN (SEQ ID
NO: 11));
and
d) HC-FR4
(WGQGTQVTVSA (SEQ ID NO: 12)).

In some embodiments of the anti-CD30 antibodies described herein, the light chain CDR sequences comprise the following:

a) CDR-L1
(KASQSVDFDGDSYMN (SEQ ID NO: 4));
b) CDR-L2
(AASNLES (SEQ ID NO: 5));
and
c) CDR-L3
(QQSNEDPWT (SEQ ID NO: 6)).

In some embodiments of the anti-CD30 antibodies described herein, the light chain FR sequences comprise the following:

a) LC-FR1
(DIVLTQSPASLAVSLGQRATISC (SEQ ID NO: 13));
b) LC-FR2
(WYQQKPGQPPKVLIY (SEQ ID NO: 14));
c) LC-FR3
(GIPARFSGSGSGTDFTLNIHPVEEEDAATYYC (SEQ ID
NO: 15));
and
d) LC-FR4
(FGGGTKLEIK (SEQ ID NO: 16)).

In some embodiments, provided herein is an anti-CD30 antibody that binds to CD30 (e.g., human CD30), wherein the antibody comprises a heavy chain variable region and a light chain variable region, wherein the antibody comprises:
(a) heavy chain variable domain comprising:
(1) an HC-FR1 comprising the amino acid sequence of SEQ ID NO:9;
(2) an CDR-H1 comprising the amino acid sequence of SEQ ID NO:1;
(3) an HC-FR2 comprising the amino acid sequence of SEQ ID NO:10;
(4) an CDR-12 comprising the amino acid sequence of SEQ ID NO:2;
(5) an HC-FR3 comprising the amino acid sequence of SEQ ID NO:11;
(6) an CDR-H3 comprising the amino acid sequence of SEQ ID NO:3; and
(7) an HC-FR4 comprising the amino acid sequence of SEQ ID NO:12,
and/or
(b) a light chain variable domain comprising:
(1) an LC-FR1 comprising the amino acid sequence of SEQ ID NO:13;
(2) an CDR-L1 comprising the amino acid sequence of SEQ ID NO:4;
(3) an LC-FR2 comprising the amino acid sequence of SEQ ID NO: 14;
(4) an CDR-L2 comprising the amino acid sequence of SEQ ID NO:5;
(5) an LC-FR3 comprising the amino acid sequence of SEQ ID NO: 15;
(6) an CDR-L3 comprising the amino acid sequence of SEQ ID NO: 6; and
(7) an LC-FR4 comprising the amino acid sequence of SEQ ID NO: 16.

In one aspect, provided herein is an anti-CD30 antibody comprising a heavy chain variable domain comprising the amino acid sequence of SEQ ID NO:7 and/or comprising a light chain variable domain comprising the amino acid sequence of SEQ ID NO:8.

In some embodiments, provided herein is an anti-CD30 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO: 1, (b) CDR-H2 comprising the amino acid sequence of SEQ ID NO:2, and (c) CDR-H3 comprising the amino acid sequence of SEQ ID NO:3.

In some embodiments, provided herein is an anti-CD30 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a light chain variable domain sequence of SEQ ID NO:8 including post-translational modifications of that sequence. In a particular embodiment, the light chain variable domain comprises one, two or three CDRs selected from: (a) CDR-H1 comprising the amino acid sequence of SEQ ID NO:4, (b) CDR-H2 comprising the amino acid sequence of SEQ ID NO:5, and (c) CDR-H3 comprising the amino acid sequence of SEQ ID NO:6.

In some embodiments, the anti-CD30 antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-CD30 antibody of the anti-CD30 antibody-drug conjugate comprises: i) a heavy chain CDR1 set out in SEQ ID NO: 1, a heavy chain CDR2 set out in SEQ ID NO: 2, a heavy chain CDR3 set out in SEQ ID NO: 3; and ii) a light chain CDR1 set out in SEQ ID NO: 4, a light chain CDR2 set out in SEQ ID NO: 5, and a light chain CDR3 set out in SEQ ID NO: 6.

In some embodiments, the anti-CD30 antibody of the anti-CD30 antibody-drug conjugate comprises: i) an amino acid sequence at least 85% identical to a heavy chain variable region set out in SEQ ID NO: 7, and ii) an amino acid sequence at least 85% identical to a light chain variable region set out in SEQ ID NO: 8.

In some embodiments, the anti-CD30 antibody of the anti-CD30 antibody-drug conjugate is a monoclonal antibody.

In some embodiments, the anti-CD30 antibody of the anti-CD30 antibody-drug conjugate is a chimeric AC10 antibody.

In some embodiments, the anti-CD30 antibody of the anti-CD30 antibody-drug conjugate is brentuximab or a biosimilar thereof. In some embodiments, the anti-CD30 antibody of the anti-CD30 antibody-drug conjugate is brentuximab.

In some embodiments, the anti-CD30 antibody is an anti-CD30 antibody or antigen-binding fragment thereof that binds to the same epitope as cAC10, e.g., the same epitope as brentuximab. In certain embodiments, the anti-CD30 antibody is an antibody that has the same CDRs as cAC10, e.g., the same CDRs as brentuximab. Antibodies that bind to the same epitope are expected to have functional properties very similar to those of cAC10 by virtue of their binding to the same epitope region of CD30. These antibodies can be readily identified based on their ability to, for example, cross-compete with cAC10 in standard CD30 binding assays such as Biacore analysis, ELISA assays, or flow cytometry.

In certain embodiments, the antibodies that cross-compete for binding to human CD30 with, or bind to the same epitope region of human CD30 as cAC10 are monoclonal antibodies. For administration to human subjects, these cross-competing antibodies can be chimeric antibodies, or can be humanized or human antibodies. Such chimeric, humanized, or human monoclonal antibodies can be prepared and isolated by methods well known in the art. Anti-CD30 antibodies usable in the methods of the disclosed disclosure also include antigen-binding fragments of the above antibodies.

Antibodies of the present invention may also be described or specified in terms of their binding affinity to CD30. Preferred binding affinities include those with a dissociation constant or Kd less than 5×10² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In one aspect of the invention, polynucleotides encoding anti-CD30 antibodies, such as those anti-CD30 antibodies described herein, are provided. In certain embodiments, vectors comprising polynucleotides encoding anti-CD30 antibodies as described herein are provided. In certain embodiments, host cells comprising such vectors are provided. In another aspect of the invention, compositions comprising anti-CD30 antibodies described herein or polynucleotides encoding anti-CD30 antibodies described herein are provided.

The antibodies also include derivatives that are modified, i.e., by the covalent attachment of any type of molecule to the antibody such that covalent attachment does not prevent the antibody from binding to CD30 or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

ii. Antibody-Drug Conjugate Structure

In some embodiments, the anti-CD30 antibody is conjugated to a therapeutic agent (e.g., an anti-CD30 antibody-drug conjugate). In some embodiments, the therapeutic agent comprises an anti-neoplastic agent (e.g., an anti-mitotic agent). In certain embodiments, the therapeutic agent is camptothecin or a functional analog thereof or a functional derivative thereof. In certain embodiments, the therapeutic agent comprises a camptothecin conjugate of Formula (IC):

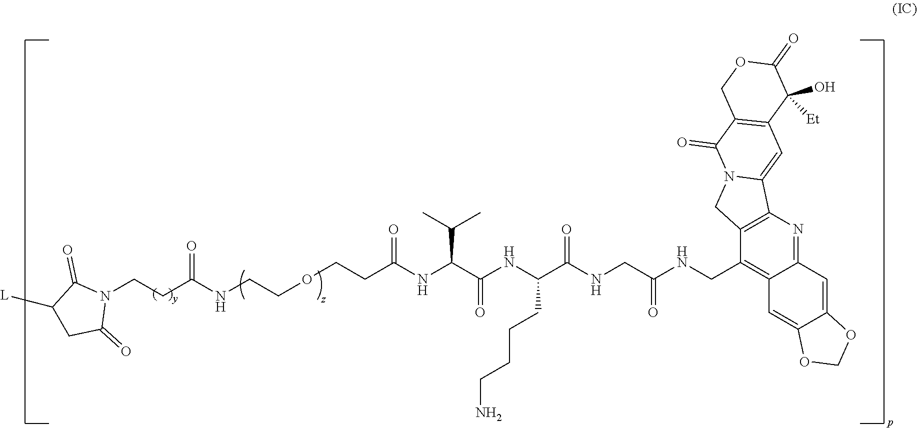

or a pharmaceutically acceptable salt thereof, wherein:
L is an anti-CD30 antibody or antigen-binding fragment thereof as described herein,
y is 1, 2, 3, or 4, or is 1 or 4,
z is an integer from 2 to 12, or is 2, 4, 8, or 12, and
p is 1-16, or is 2, 3, 4, 5, 6, 7, 8, 9, or 10, or is 2, 4 or 8.

In some embodiments, y is 1, 2, 3, or 4. In some embodiments, y is 1 or 2. In some embodiments, y is 1 or 3. In some embodiments, y is 1 or 4. In some embodiments, y is 2 or 3. In some embodiments, y is 2 or 4. In some embodiments, y is 3 or 4. In some embodiments, y is 1, 2, or 3. In some embodiments, y is 2, 3, or 4. In some embodiments, y is 1. In some embodiments, y is 2. In some embodiments, y is 3. In some embodiments, y is 4.

In some embodiments, when y is 1, provided herein is a formulation containing a camptothecin conjugate of Formula (I):

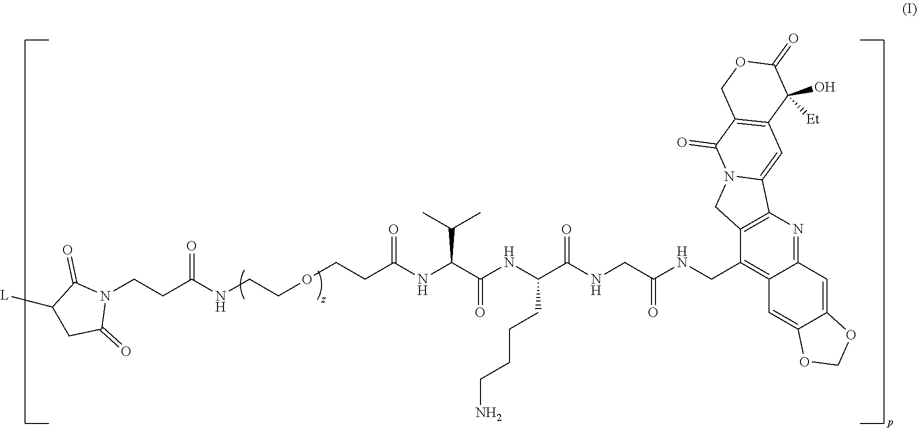

or a pharmaceutically acceptable salt thereof, wherein:
L is an antibody,
z is an integer from 2 to 12, or is 2, 4, 8, or 12, and
p is 1-16, or is 2, 3, 4, 5, 6, 7, 8, 9, or 10, or is 2, 4 or 8.

It is understood that each description of L can be combined with each description of y the same as if each and every combination were specifically and individually listed. For example, in some embodiments, y is 1 or 4; and L is cAC10. As another example, in some embodiments, y is 1; and L is an anti-CD30 antibody comprising CDR-H1, CDR-H2, CDR-H3, CDR-L1, CDR-L2, and CDR-L3 comprising the amino acid sequences of SEQ ID NOs: 1, 2, 3, 4, 5, and 6, respectively.

In some embodiments, z is an integer from 2 to 12, from 2 to 11, from 2 to 10, from 2 to 9, from 2 to 8, from 2 to 7, from 2 to 6, from 2 to 5, from 2 to 4, from 2 to 3, from 3 to 12, from 3 to 11, from 3 to 10, from 3 to 9, from 3 to 8, from 3 to 7, from 3 to 6, from 3 to 5, from 3 to 4, from 4 to 12, from 4 to 11, from 4 to 10, from 4 to 9, from 4 to 8, from 4 to 7, from 4 to 6, from 4 to 5, from 5 to 12, from 5 to 11, from 5 to 10, from 5 to 9, from 5 to 8, from 5 to 7, from 5 to 6, from 6 to 12, from 6 to 11, from 6 to 10, from 6 to 9, from 6 to 8, from 6 to 7, from 7 to 12, from 7 to 11, from 7 to 10, from 7 to 9, from 7 to 8, from 8 to 12, from 8 to 11, from 8 to 10, from 8 to 9, from 9 to 12, from 9 to 11, from 9 to 10, from 10 to 12, from 10 to 11, or from 11 to 12. In some embodiments, z is 2, 3, 4, 5, 6, 7, 8, 9, 10, 11, or 12. In some embodiments, z is 2, 4, 6, 8, 10, or 12. In some embodiments, z is 2, 4, 8, or 12. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. It is understood that each description of z can be combined with each description of y and/or L the same as if each and every combination were specifically and individually listed. For example, in some embodiments, L is cAC10; and z is 2, 4, or 8. As another example, in some embodiments, L is an anti-CD30 antibody comprising CDR-H1, CDR-H2, CDR-H3, CDR-L1, CDR-L2, and CDR-L3 comprising the amino acid sequences of SEQ ID NOs: 1, 2, 3, 4, 5, and 6, respectively; and z is 8. As another example, in some embodiments, y is 1 or 4; and z is 2, 4, or 8. As another example, in some embodiments, y is 1; and z is 8. As another example, in some embodiments, L is cAC10; y is 1 or 4; and z is 2, 4, or 8. As another example, in some embodiments, L is an anti-CD30 antibody comprising CDR-H1, CDR-H2, CDR-H3, CDR-L1, CDR-L2, and CDR-L3 comprising the amino acid sequences of SEQ ID NOs: 1, 2, 3, 4, 5, and 6, respectively; y is 1; and z is 8

In some embodiments, the subscript p represents the number of drug linker moieties on an antibody of an individual camptothecin conjugate and is an integer preferably ranging from 1 to 16, 1 to 12, 1 to 10, or 1 to 8. Individual camptothecin conjugate can be also be referred to as a camptothecin conjugate compound. In some embodiments, p is an integer from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, from 1 to 2, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, from 2 to 3, from 3 to 16, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4, from 4 to 16, from 4 to 15, from 4 to 14, from 4 to 13, from 4 to 12, from 4 to 11, from 4 to 10, from 4 to 9, from 4 to 8, from 4 to 7, from 4 to 6, from 4 to 5, from 5 to 16, from 5 to 15, from 5 to 14, from 5 to 13, from 5 to 12, from 5 to 11, from 5 to 10, from 5 to 9, from 5 to 8, from 5 to 7, from 5 to 6, from 6 to 16, from 6 to 15, from 6 to 14, from 6 to 13, from 6 to 12, from 6 to 11, from 6 to 10, from 6 to 9, from 6 to 8, from 6 to 7, from 7 to 16, from 7 to 15, from 7 to 14, from 7 to 13, from 7 to 12, from 7 to 11, from 7 to 10, from 7 to 9, from 7 to 8, from 8 to 16, from 8 to 15, from 8 to 14, from 8 to 13, from 8 to 12, from 8 to 11, from 8 to 10, from 8 to 9, from 9 to 16, from 9 to 15, from 9 to 14, from 9 to 13, from 9 to 12, from 9 to 11, from 9 to 10, from 10 to 16, from 10 to 15, from 10 to 14, from 10 to 13, from 10 to 12, from 10 to 11, from 11 to 16, from 11 to 15, from 11 to 14, from 11 to 13, from 11 to 12, from 12 to 16, from 12 to 15, from 12 to 14, from 12 to 13, from 13 to 16, from 13 to 15, from 13 to 14, from 14 to 16, from 14 to 15, or from 15 to 16. In some embodiments, p is 1, 2, 3, 4, 5, 6, 7, 8, 9, 10, 11, 12, 13, 14, 15, or 16. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 13. In some embodiments, p is 14. In some embodiments, p is 15. In some embodiments, p is 16.

In one aspect, one group of embodiments contains a population of individual camptothecin conjugates substantially identical except for the number of drug-linkers bound to each antibody. The population can be described by the average number of drug-linkers bound to the antibody of the camptothecin conjugate (e.g., the Drug-Antibody Ratio (“DAR”)). In that group of embodiments, the average is a number ranging from 1 to about 16, 1 to about 12, 1 to about 10, or 1 to about 8, from 2 to about 16, 2 to about 12, 2 to about 10, or 2 to about 8. In some aspects, the average is about 2. In some aspects, the average is about 4. In some aspects, the average is about 8. In some aspects, the average is about 16. In some aspects, the average is 2. In some aspects, the average is 4. In some aspects, the average is 8. In some aspects, the average is 16. In some aspects, the population can be described by the drug loading of the predominate ADC in the composition.

In some aspects, conjugation will be via the interchain disulfides and there will from 1 to about 8 drug-linkers conjugated to an antibody. In some aspects, conjugation will be via an introduced cysteine residue as well as interchain disulfides and there will be from 1 to 10 or 1 to 12 or 1 to 14 or 1 to 16 drug-linkers conjugated to an antibody. In some aspects, conjugation will be via an introduced cysteine residue and there will be 2 or 4 drug-linkers conjugated to an antibody.

It is understood that each description of p can be combined with each description of L, y, and/or z the same as if each and every combination were specifically and individually listed. For example, in some embodiments, L is cAC10; z is 2, 4, or 8; and p is 8. As another example, in some embodiments, L is an anti-CD30 antibody comprising CDR-H1, CDR-H2, CDR-H3, CDR-L1, CDR-L2, and CDR-L3 comprising the amino acid sequences of SEQ ID NOs: 1, 2, 3, 4, 5, and 6, respectively; z is 8; and p is 8. As another example, in some embodiments, L is cAC10; y is 1 or 4; z is 2, 4, or 8; and p is 8. As another example, in some embodiments, L is an anti-CD30 antibody comprising CDR-H1, CDR-H2, CDR-H3, CDR-L1, CDR-L2, and CDR-L3 comprising the amino acid sequences of SEQ ID NOs: 1, 2, 3, 4, 5, and 6, respectively; y is 1; z is 8; and p is 8.

In one embodiment, the antibody-drug conjugate is an antibody-drug conjugate of Formula (IC):

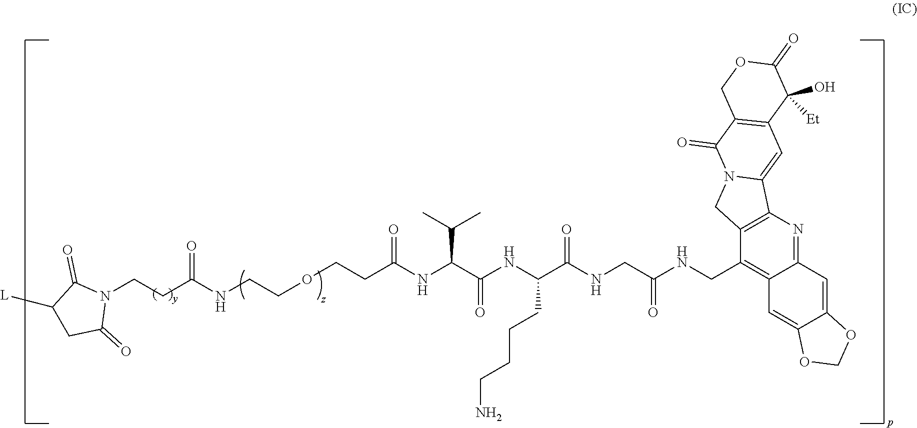

or a pharmaceutically acceptable salt thereof, wherein:
L is the anti-CD30 antibody brentuximab,
y is 1,
z is 8, and
p is 8, which is also known as SGN-CD30C. The preparation of SGN-CD30C is described in WO 2019/195665, incorporated herein by reference.

In some embodiments, the antibody-drug conjugate is an antibody-drug conjugate described in WO 2019/195665, incorporated herein by reference.

In some embodiments, the antibody-drug conjugate is an antibody-drug conjugate described in WO 2019/236954, incorporated herein by reference.

In some embodiments, the antibody-drug conjugate is an antibody-drug conjugate described in PCT/US20/54137, incorporated herein by reference.

In one embodiment, the antibody-drug conjugate is SGN-CD30C or a biosimilar thereof. In one embodiment, the antibody-drug conjugate is SGN-CD30C.

B. Methods of Treatment

The invention provides a method of modulating the immune response in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. The invention also provides a method of decreasing the number of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the number of CD30⁺ Treg cells is decreased relative to the number of CD30⁺ Treg cells in the subject prior to administration of the antibody-drug conjugate. The invention also provides a method of decreasing the activity of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the decrease in the activity of CD30⁺ Treg cells is relative to the activity of CD30⁺ Treg cells in the subject prior to administration of the antibody-drug conjugate. In some embodiments, the CD30+ Treg cells are inducible T regulatory (iTreg) cells. In some embodiments, the CD30+ Treg cells are peripheral blood T regulatory (pbTreg) cells. In some embodiments, the anti-CD30 antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6. In some embodiments, the anti-CD30 antibody or antigen-binding fragment thereof of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody or antigen-binding fragment thereof of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8. In some embodiments, the anti-CD30 antibody or antigen-binding fragment thereof of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8. In some embodiments, the anti-CD30 antibody or antigen-binding fragment thereof of the antibody-drug conjugate comprises a heavy chain variable region comprising the amino acid sequence of SEQ ID NO: 7 and a light chain variable region comprising the amino acid sequence of SEQ ID NO: 8. In some embodiments, the anti-CD30 antibody or antigen-binding fragment thereof of the antibody-drug conjugate is cAC10. In some embodiments, the antibody-drug conjugate is SGN-CD30C. In some embodiments, the subject has cancer. In some embodiments, the cancer is a hematologic cancer. In some embodiments, the cancer is selected from the group consisting of Hodgkin lymphoma, non-Hodgkin lymphoma, anaplastic large cell lymphoma, peripheral T-cell lymphoma, or mycosis fungoides. In some embodiments, the cancer is Hodgkin lymphoma. In some embodiments, the Hodgkin lymphoma is classical Hodgkin lymphoma (cHL). In some embodiments, the cancer is non-Hodgkin lymphoma. In some embodiments, the non-Hodgkin lymphoma is diffuse large B-cell lymphoma (DLBCL). In some embodiments, the DLBCL is germinal-center B-cell like (GCB). In some embodiments, the DLBCL is non-GCB. In some embodiments, the cancer is anaplastic large cell lymphoma. In some embodiments, the anaplastic large cell lymphoma is systemic anaplastic large cell lymphoma. In some embodiments, the anaplastic large cell lymphoma is primary cutaneous anaplastic large cell lymphoma. In some embodiments, the non-Hodgkin lymphoma is a mature T-cell lymphoma. In some embodiments, the non-Hodgkin lymphoma is cutaneous T-cell lymphoma (CTCL). In some embodiments, the cancer is peripheral T-cell lymphoma. In some embodiments, the peripheral T-cell lymphoma is angioimmunoblastic T-cell lymphoma. In some embodiments, the cancer is mycosis fungoides. In some embodiments, the cancer is a non-hematologic cancer. In some embodiments, the non-hematologic cancer is a carcinoma. In some embodiments, the non-hematologic cancer is a sarcoma. In some embodiments, the non-hematologic cancer is a solid tumor. In some embodiments, the cancer is a CD30+ cancer, while in other embodiments, the cancer is a CD30-cancer. In some embodiments, the cancer is an advanced stage cancer. In some embodiments, the advanced stage cancer is a stage 3 or stage 4 cancer. In some embodiments, the advanced stage cancer is metastatic cancer. In some embodiments, the subject has been previously treated with one or more therapeutic agents and did not respond to the treatment. In some embodiments, the subject has been previously treated with one or more therapeutic agents and relapsed after the treatment. In some embodiments, the subject has been previously treated with one or more therapeutic agents and has experienced disease progression during treatment. In some embodiments, the subject has previously received allogenic stem cell transplant to treat the cancer. In some embodiments, the subject has previously received autologous stem cell transplant to treat the cancer. In some embodiments, the subject relapsed following stem cell transplant. In some embodiments, the subject has previously received CAR-T therapy. In some embodiments, the subject relapsed after CAR-T therapy. In some embodiments, the cancer is recurrent cancer. In some embodiments, the subject has not previously been treated for the cancer. In some embodiments, the subject has not been previously treated with an antibody-drug conjugate that binds to CD30. In some embodiments, at least 1% of the cancer cells in the subject express CD30. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells in the subject express CD30. In a particular embodiment, the subject is a human. In certain embodiments, the subject is further administered granulocyte colony-stimulating factor (G-CSF). In certain embodiments, the G-CSF is administered prophylactically. In certain embodiments, the G-CSF is administered 1 to 3 days after the administration of the first anti-CD30 antibody-drug conjugate and/or the second anti-CD30 antibody-drug conjugate. In certain embodiments, the G-CSF is administered 1 day after the administration of the first anti-CD30 antibody-drug conjugate and/or the second anti-CD30 antibody-drug conjugate. In certain embodiments, the G-CSF is administered 2 days after the administration of the first anti-CD30 antibody-drug conjugate and/or the second anti-CD30 antibody-drug conjugate. In certain embodiments, the G-CSF is administered 3 days after the administration of the first anti-CD30 antibody-drug conjugate and/or the second anti-CD30 antibody-drug conjugate. In certain embodiments, the G-CSF is recombinant human G-CSF. In certain embodiments, the GCSF is filgrastim (NEUPOGEN®). In certain embodiments, the G-CSF is PEG-filgrastim (NEULASTA®). In certain embodiments, the G-CSF is lenograstim (GRANOCYTE®). In certain embodiments, the G-CSF is tbo-filgrastim (GRANIX®).

C. Routes of Administration

An anti-CD30 antibody-drug conjugated to a camptothecin or a functional analog thereof or a functional derivative thereof as described herein can be administered by any suitable route and mode. Suitable routes of administering an anti-CD30 antibody-drug conjugated to a camptothecin or a functional analog thereof or a functional derivative thereof as described herein are well known in the art and may be selected by those of ordinary skill in the art. In one embodiment, an anti-CD30 antibody-drug conjugated to a camptothecin or a functional analog thereof or a functional derivative thereof as described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-CD30 antibody-drug conjugated to a camptothecin or a functional analog thereof or a functional derivative thereof as described herein is intravenous infusion. In some embodiments, the route of administration of an anti-CD30 antibody-drug conjugated to a camptothecin or a functional analog thereof or a functional derivative thereof as described herein is subcutaneous injection.

D. Dosage and Frequency of Administration

In one aspect, the present invention provides for methods of treating a subject with a particular dose of an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein with particular frequencies.

In some embodiments, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered to a subject in a dose ranging from about 0.01 mg/kg to about 100 mg/kg of the subject's body weight or from 1.0 mg/kg to 5.0 mg/kg of the subject's body weight. In some embodiments, the dose administered to a subject is between about 0.01 mg/kg to about 15 mg/kg of the subject's body weight. In some embodiments, the dose administered to a subject is between about 0.1 mg/kg and about 15 mg/kg of the subject's body weight. In some embodiments, the dose administered to a subject is between about 0.1 mg/kg and about 20 mg/kg of the subject's body weight. In some embodiments, the dose administered is between about 0.1 mg/kg to about 5 mg/kg or about 0.1 mg/kg to about 10 mg/kg of the subject's body weight. In some embodiments, the dose administered is between about 1 mg/kg to about 15 mg/kg of the subject's body weight. In some embodiments, the dose administered is between about 1 mg/kg to about 10 mg/kg of the subject's body weight. In some embodiments, the dose administered is between about 0.1 mg/kg to about 4 mg/kg of the subject's body weight. In some embodiments, the dose administered is between about 0.1 mg/kg to about 3.2 mg/kg of the subject's body weight. In some embodiments, the dose administered is between about 0.1 mg/kg to about 2.7 mg/kg of the subject's body weight.

In some embodiments, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered to a subject in a dose ranging from about 0.05 mg/kg to about 5 mg/kg of the subject's body weight. In certain embodiments, the dose is about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, about 1.2 mg/kg, about 1.25 mg/kg, about 1.3 mg/kg, about 1.35 mg/kg, about 1.4 mg/kg, about 1.45 mg/kg, about 1.5 mg/kg, about 1.55 mg/kg, about 1.6 mg/kg, about 1.65 mg/kg, about 1.7 mg/kg, about 1.75 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg of the subject's body weight. In one embodiment, the dose is about 0.1 mg/kg of the subject's body weight. In one embodiment, the dose is about 0.5 mg/kg of the subject's body weight. In certain embodiments, the dose is 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.05 mg/kg, 1.1 mg/kg, 1.15 mg/kg, 1.2 mg/kg, 1.25 mg/kg, 1.3 mg/kg, 1.35 mg/kg, 1.4 mg/kg, 1.45 mg/kg, 1.5 mg/kg, 1.55 mg/kg, 1.6 mg/kg, 1.65 mg/kg, 1.7 mg/kg, 1.75 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg of the subject's body weight. In one embodiment, the dose is 0.1 mg/kg of the subject's body weight. In one embodiment, the dose is 0.5 mg/kg of the subject's body weight. In one embodiment, the dose is 0.4 mg/kg of the subject's body weight. In one embodiment, the dose is 0.1 mg/kg of the subject's body weight and the anti-CD30 antibody-drug conjugate is SGN-CD30C. In one embodiment, the dose is 0.5 mg/kg of the subject's body weight and the anti-CD30 antibody-drug conjugate is SGN-CD30C. In some embodiments, for a subject weighing more than 100 kg, the dose of the anti-CD30 antibody-drug conjugate administered is the amount that would be administered if the subject weighed 100 kg. In some embodiments, for a subject weighing more than 100 kg, the dose of the anti-CD30 antibody-drug conjugate administered is 10 mg. In some embodiments, for a subject weighing more than 100 kg, the dose of the anti-CD30 antibody-drug conjugate administered is 50 mg.

In some embodiments of the methods or uses or product for uses provided herein, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered to the subject once about every 1 to 4 weeks. In certain embodiments, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once about every 1 week, once about every 2 weeks, once about every 3 weeks or once about every 4 weeks. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once about every 1 week. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once about every 2 weeks. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once about every 3 weeks. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once about every 4 weeks. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once every 1 week. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once every 2 weeks. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once every 3 weeks. In one embodiment, an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is administered once every 4 weeks. In some embodiments, the dose is about 0.05 mg/kg and is administered once about every 1 week. In some embodiments, the dose is about 0.05 mg/kg and is administered once about every 2 weeks. In some embodiments, the dose is about 0.05 mg/kg and is administered once about every 3 weeks. In some embodiments, the dose is about 0.05 mg/kg and is administered once about every 4 weeks. In some embodiments, the dose is about 0.1 mg/kg and is administered once about every 1 week. In some embodiments, the dose is about 0.1 mg/kg and is administered once about every 2 weeks. In some embodiments, the dose is about 0.1 mg/kg and is administered once about every 3 weeks. In some embodiments, the dose is about 0.1 mg/kg and is administered once about every 4 weeks. In some embodiments, the dose is about 0.15 mg/kg and is administered once about every 1 week. In some embodiments, the dose is about 0.15 mg/kg and is administered once about every 2 weeks. In some embodiments, the dose is about 0.15 mg/kg and is administered once about every 3 weeks. In some embodiments, the dose is about 0.15 mg/kg and is administered once about every 4 weeks. In some embodiments, the dose is about 0.2 mg/kg and is administered once about every 1 week. In some embodiments, the dose is about 0.2 mg/kg and is administered once about every 2 weeks. In some embodiments, the dose is about 0.2 mg/kg and is administered once about every 3 weeks. In some embodiments, the dose is about 0.2 mg/kg and is administered once about every 4 weeks. In some embodiments, the dose is about 0.25 mg/kg and is administered once about every 1 week. In some embodiments, the dose is about 0.25 mg/kg and is administered once about every 2 weeks. In some embodiments, the dose is about 0.25 mg/kg and is administered once about every 3 weeks. In some embodiments, the dose is about 0.25 mg/kg and is administered once about every 4 weeks. In some embodiments, the dose is about 0.3 mg/kg and is administered once about every 1 week. In some embodiments, the dose is about 0.3 mg/kg and is administered once about every 2 weeks. In some embodiments, the dose is about 0.3 mg/kg and is administered once about every 3 weeks. In some embodiments, the dose is about 0.3 mg/kg and is administered once about every 4 weeks. In some embodiments, the dose is about 0.35 mg/kg and is administered once about every 1 week. In some embodiments, the dose is about 0.35 mg/kg and is administered once about every 2 weeks. In some embodiments, the dose is about 0.35 mg/kg and is administered once about every 3 weeks. In some embodiments, the dose is about 0.35 mg/kg and is administered once about every 4 weeks. In some embodiments, the dose is about 0.4 mg/kg and is administered once about every 1 week. In some embodiments, the dose is about 0.4 mg/kg and is administered once about every 2 weeks. In some embodiments, the dose is about 0.4 mg/kg and is administered once about every 3 weeks. In some embodiments, the dose is about 0.4 mg/kg and is administered once about every 4 weeks. In some embodiments, the dose is about 0.45 mg/kg and is administered once about every 1 week. In some embodiments, the dose is about 0.45 mg/kg and is administered once about every 2 weeks. In some embodiments, the dose is about 0.45 mg/kg and is administered once about every 3 weeks. In some embodiments, the dose is about 0.45 mg/kg and is administered once about every 4 weeks. In some embodiments, the dose is about 0.5 mg/kg and is administered once about every 1 week. In some embodiments, the dose is about 0.5 mg/kg and is administered once about every 2 weeks. In some embodiments, the dose is about 0.5 mg/kg and is administered once about every 3 weeks. In some embodiments, the dose is about 0.5 mg/kg and is administered once about every 4 weeks. In some embodiments, the dose is about 0.55 mg/kg and is administered once about every 1 week. In some embodiments, the dose is about 0.55 mg/kg and is administered once about every 2 weeks. In some embodiments, the dose is about 0.55 mg/kg and is administered once about every 3 weeks. In some embodiments, the dose is about 0.55 mg/kg and is administered once about every 4 weeks. In some embodiments, the dose is about 0.6 mg/kg and is administered once about every 1 week. In some embodiments, the dose is about 0.6 mg/kg and is administered once about every 2 weeks. In some embodiments, the dose is about 0.6 mg/kg and is administered once about every 3 weeks. In some embodiments, the dose is about 0.6 mg/kg and is administered once about every 4 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.7 mg/kg and is administered once about every 1 week. In some embodiments, the dose is about 0.7 mg/kg and is administered once about every 2 weeks. In some embodiments, the dose is about 0.7 mg/kg and is administered once about every 3 weeks. In some embodiments, the dose is about 0.7 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.8 mg/kg and is administered once about every 1 week. In some embodiments, the dose is about 0.8 mg/kg and is administered once about every 2 weeks. In some embodiments, the dose is about 0.8 mg/kg and is administered once about every 3 weeks. In some embodiments, the dose is about 0.8 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 0.95 mg/kg and is administered once about every 1 week. In some embodiments, the dose is about 0.95 mg/kg and is administered once about every 2 weeks. In some embodiments, the dose is about 0.95 mg/kg and is administered once about every 3 weeks. In some embodiments, the dose is about 0.95 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.05 mg/kg and is administered once about every 1 week. In some embodiments, the dose is about 1.05 mg/kg and is administered once about every 2 weeks. In some embodiments, the dose is about 1.05 mg/kg and is administered once about every 3 weeks. In some embodiments, the dose is about 1.05 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.15 mg/kg and is administered once about every 1 week. In some embodiments, the dose is about 1.15 mg/kg and is administered once about every 2 weeks. In some embodiments, the dose is about 1.15 mg/kg and is administered once about every 3 weeks. In some embodiments, the dose is about 1.15 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.25 mg/kg and is administered once about every 1 week. In some embodiments, the dose is about 1.25 mg/kg and is administered once about every 2 weeks. In some embodiments, the dose is about 1.25 mg/kg and is administered once about every 3 weeks. In some embodiments, the dose is about 1.25 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.35 mg/kg and is administered once about every 1 week. In some embodiments, the dose is about 1.35 mg/kg and is administered once about every 2 weeks. In some embodiments, the dose is about 1.35 mg/kg and is administered once about every 3 weeks. In some embodiments, the dose is about 1.35 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.45 mg/kg and is administered once about every 1 week. In some embodiments, the dose is about 1.45 mg/kg and is administered once about every 2 weeks. In some embodiments, the dose is about 1.45 mg/kg and is administered once about every 3 weeks. In some embodiments, the dose is about 1.45 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.55 mg/kg and is administered once about every 1 week. In some embodiments, the dose is about 1.55 mg/kg and is administered once about every 2 weeks. In some embodiments, the dose is about 1.55 mg/kg and is administered once about every 3 weeks. In some embodiments, the dose is about 1.55 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.65 mg/kg and is administered once about every 1 week. In some embodiments, the dose is about 1.65 mg/kg and is administered once about every 2 weeks. In some embodiments, the dose is about 1.65 mg/kg and is administered once about every 3 weeks. In some embodiments, the dose is about 1.65 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.75 mg/kg and is administered once about every 1 week. In some embodiments, the dose is about 1.75 mg/kg and is administered once about every 2 weeks. In some embodiments, the dose is about 1.75 mg/kg and is administered once about every 3 weeks. In some embodiments, the dose is about 1.75 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is about 2.2 mg/kg and is administered once about every 1 week. In some embodiments, the dose is about 2.2 mg/kg and is administered once about every 2 weeks. In some embodiments, the dose is about 2.2 mg/kg and is administered once about every 3 weeks. In some embodiments, the dose is about 2.2 mg/kg and is administered once about every 4 weeks. In some embodiments, the dose is about 2.3 mg/kg and is administered once about every 1 week. In some embodiments, the dose is about 2.3 mg/kg and is administered once about every 2 weeks. In some embodiments, the dose is about 2.3 mg/kg and is administered once about every 3 weeks. In some embodiments, the dose is about 2.3 mg/kg and is administered once about every 4 weeks. In some embodiments, the dose is about 2.4 mg/kg and is administered once about every 1 week. In some embodiments, the dose is about 2.4 mg/kg and is administered once about every 2 weeks. In some embodiments, the dose is about 2.4 mg/kg and is administered once about every 3 weeks. In some embodiments, the dose is about 2.4 mg/kg and is administered once about every 4 weeks. In some embodiments, the dose is about 2.5 mg/kg and is administered once about every 1 week. In some embodiments, the dose is about 2.5 mg/kg and is administered once about every 2 weeks. In some embodiments, the dose is about 2.5 mg/kg and is administered once about every 3 weeks. In some embodiments, the dose is about 2.5 mg/kg and is administered once about every 4 weeks. In some embodiments, the dose is about 2.6 mg/kg and is administered once about every 1 week. In some embodiments, the dose is about 2.7 mg/kg and is administered once about every 2 weeks. In some embodiments, the dose is about 2.7 mg/kg and is administered once about every 3 weeks. In some embodiments, the dose is about 2.7 mg/kg and is administered once about every 4 weeks. In some embodiments, the dose is about 2.8 mg/kg and is administered once about every 1 week. In some embodiments, the dose is about 2.8 mg/kg and is administered once about every 2 weeks. In some embodiments, the dose is about 2.8 mg/kg and is administered once about every 3 weeks. In some embodiments, the dose is about 2.8 mg/kg and is administered once about every 4 weeks. In some embodiments, the dose is about 2.9 mg/kg and is administered once about every 1 week. In some embodiments, the dose is about 2.9 mg/kg and is administered once about every 2 weeks. In some embodiments, the dose is about 2.9 mg/kg and is administered once about every 3 weeks. In some embodiments, the dose is about 2.9 mg/kg and is administered once about every 4 weeks. In some embodiments, the dose is about 3.0 mg/kg and is administered once about every 1 week. In some embodiments, the dose is about 3.0 mg/kg and is administered once about every 2 weeks. In some embodiments, the dose is about 3.0 mg/kg and is administered once about every 3 weeks. In some embodiments, the dose is about 3.0 mg/kg and is administered once about every 4 weeks. In some embodiments, the dose is about 3.1 mg/kg and is administered once about every 1 week. In some embodiments, the dose is about 3.1 mg/kg and is administered once about every 2 weeks. In some embodiments, the dose is about 3.1 mg/kg and is administered once about every 3 weeks. In some embodiments, the dose is about 3.1 mg/kg and is administered once about every 4 weeks. In some embodiments, the dose is about 3.2 mg/kg and is administered once about every 1 week. In some embodiments, the dose is about 3.2 mg/kg and is administered once about every 2 weeks. In some embodiments, the dose is about 3.2 mg/kg and is administered once about every 3 weeks. In some embodiments, the dose is about 3.2 mg/kg and is administered once about every 4 weeks. In some embodiments, the dose is about 3.3 mg/kg and is administered once about every 1 week. In some embodiments, the dose is about 3.3 mg/kg and is administered once about every 2 weeks. In some embodiments, the dose is about 3.3 mg/kg and is administered once about every 3 weeks. In some embodiments, the dose is about 3.3 mg/kg and is administered once about every 4 weeks. In some embodiments, the dose is about 3.4 mg/kg and is administered once about every 1 week. In some embodiments, the dose is about 3.4 mg/kg and is administered once about every 2 weeks. In some embodiments, the dose is about 3.4 mg/kg and is administered once about every 3 weeks. In some embodiments, the dose is about 3.4 mg/kg and is administered once about every 4 weeks. In some embodiments, the dose is about 3.5 mg/kg and is administered once about every 1 week. In some embodiments, the dose is about 3.5 mg/kg and is administered once about every 2 weeks. In some embodiments, the dose is about 3.5 mg/kg and is administered once about every 3 weeks. In some embodiments, the dose is about 3.5 mg/kg and is administered once about every 4 weeks. In some embodiments, the dose is about 3.6 mg/kg and is administered once about every 1 week. In some embodiments, the dose is about 3.7 mg/kg and is administered once about every 2 weeks. In some embodiments, the dose is about 3.7 mg/kg and is administered once about every 3 weeks. In some embodiments, the dose is about 3.7 mg/kg and is administered once about every 4 weeks. In some embodiments, the dose is about 3.8 mg/kg and is administered once about every 1 week. In some embodiments, the dose is about 3.8 mg/kg and is administered once about every 2 weeks. In some embodiments, the dose is about 3.8 mg/kg and is administered once about every 3 weeks. In some embodiments, the dose is about 3.8 mg/kg and is administered once about every 4 weeks. In some embodiments, the dose is about 3.9 mg/kg and is administered once about every 1 week. In some embodiments, the dose is about 3.9 mg/kg and is administered once about every 2 weeks. In some embodiments, the dose is about 3.9 mg/kg and is administered once about every 3 weeks. In some embodiments, the dose is about 3.9 mg/kg and is administered once about every 4 weeks. In some embodiments, the dose is about 4.0 mg/kg and is administered once about every 1 week. In some embodiments, the dose is about 4.0 mg/kg and is administered once about every 2 weeks. In some embodiments, the dose is about 4.0 mg/kg and is administered once about every 3 weeks. In some embodiments, the dose is about 4.0 mg/kg and is administered once about every 4 weeks. In some embodiments, the dose is about 4.1 mg/kg and is administered once about every 1 week. In some embodiments, the dose is about 4.1 mg/kg and is administered once about every 2 weeks. In some embodiments, the dose is about 4.1 mg/kg and is administered once about every 3 weeks. In some embodiments, the dose is about 4.1 mg/kg and is administered once about every 4 weeks. In some embodiments, the dose is about 4.2 mg/kg and is administered once about every 1 week. In some embodiments, the dose is about 4.2 mg/kg and is administered once about every 2 weeks. In some embodiments, the dose is about 4.2 mg/kg and is administered once about every 3 weeks. In some embodiments, the dose is about 4.2 mg/kg and is administered once about every 4 weeks. In some embodiments, the dose is about 4.3 mg/kg and is administered once about every 1 week. In some embodiments, the dose is about 4.3 mg/kg and is administered once about every 2 weeks. In some embodiments, the dose is about 4.3 mg/kg and is administered once about every 3 weeks. In some embodiments, the dose is about 4.3 mg/kg and is administered once about every 4 weeks. In some embodiments, the dose is about 4.4 mg/kg and is administered once about every 1 week. In some embodiments, the dose is about 4.4 mg/kg and is administered once about every 2 weeks. In some embodiments, the dose is about 4.4 mg/kg and is administered once about every 3 weeks. In some embodiments, the dose is about 4.4 mg/kg and is administered once about every 4 weeks. In some embodiments, the dose is about 4.5 mg/kg and is administered once about every 1 week. In some embodiments, the dose is about 4.5 mg/kg and is administered once about every 2 weeks. In some embodiments, the dose is about 4.5 mg/kg and is administered once about every 3 weeks. In some embodiments, the dose is about 4.5 mg/kg and is administered once about every 4 weeks. In some embodiments, the dose is about 4.6 mg/kg and is administered once about every 1 week. In some embodiments, the dose is about 4.7 mg/kg and is administered once about every 2 weeks. In some embodiments, the dose is about 4.7 mg/kg and is administered once about every 3 weeks. In some embodiments, the dose is about 4.7 mg/kg and is administered once about every 4 weeks. In some embodiments, the dose is about 4.8 mg/kg and is administered once about every 1 week. In some embodiments, the dose is about 4.8 mg/kg and is administered once about every 2 weeks. In some embodiments, the dose is about 4.8 mg/kg and is administered once about every 3 weeks. In some embodiments, the dose is about 4.8 mg/kg and is administered once about every 4 weeks. In some embodiments, the dose is about 4.9 mg/kg and is administered once about every 1 week. In some embodiments, the dose is about 4.9 mg/kg and is administered once about every 2 weeks. In some embodiments, the dose is about 4.9 mg/kg and is administered once about every 3 weeks. In some embodiments, the dose is about 4.9 mg/kg and is administered once about every 4 weeks. In some embodiments, the dose is about 5.0 mg/kg and is administered once about every 1 week. In some embodiments, the dose is about 5.0 mg/kg and is administered once about every 2 weeks. In some embodiments, the dose is about 5.0 mg/kg and is administered once about every 3 weeks. In some embodiments, the dose is about 5.0 mg/kg and is administered once about every 4 weeks. In some embodiments, the dose is 0.05 mg/kg and is administered once about every 1 week. In some embodiments, the dose is 0.05 mg/kg and is administered once about every 2 weeks. In some embodiments, the dose is 0.05 mg/kg and is administered once about every 3 weeks. In some embodiments, the dose is 0.05 mg/kg and is administered once about every 4 weeks. In some embodiments, the dose is 0.1 mg/kg and is administered once about every 1 week. In some embodiments, the dose is 0.1 mg/kg and is administered once about every 2 weeks. In some embodiments, the dose is 0.1 mg/kg and is administered once about every 3 weeks. In some embodiments, the dose is 0.1 mg/kg and is administered once about every 4 weeks. In some embodiments, the dose is 0.15 mg/kg and is administered once about every 1 week. In some embodiments, the dose is 0.15 mg/kg and is administered once about every 2 weeks. In some embodiments, the dose is 0.15 mg/kg and is administered once about every 3 weeks. In some embodiments, the dose is 0.15 mg/kg and is administered once about every 4 weeks. In some embodiments, the dose is 0.2 mg/kg and is administered once about every 1 week. In some embodiments, the dose is 0.2 mg/kg and is administered once about every 2 weeks. In some embodiments, the dose is 0.2 mg/kg and is administered once about every 3 weeks. In some embodiments, the dose is 0.2 mg/kg and is administered once about every 4 weeks. In some embodiments, the dose is 0.25 mg/kg and is administered once about every 1 week. In some embodiments, the dose is 0.25 mg/kg and is administered once about every 2 weeks. In some embodiments, the dose is 0.25 mg/kg and is administered once about every 3 weeks. In some embodiments, the dose is 0.25 mg/kg and is administered once about every 4 weeks. In some embodiments, the dose is 0.3 mg/kg and is administered once about every 1 week. In some embodiments, the dose is 0.3 mg/kg and is administered once about every 2 weeks. In some embodiments, the dose is 0.3 mg/kg and is administered once about every 3 weeks. In some embodiments, the dose is 0.3 mg/kg and is administered once about every 4 weeks. In some embodiments, the dose is 0.35 mg/kg and is administered once about every 1 week. In some embodiments, the dose is 0.35 mg/kg and is administered once about every 2 weeks. In some embodiments, the dose is 0.35 mg/kg and is administered once about every 3 weeks. In some embodiments, the dose is 0.35 mg/kg and is administered once about every 4 weeks. In some embodiments, the dose is 0.4 mg/kg and is administered once about every 1 week. In some embodiments, the dose is 0.4 mg/kg and is administered once about every 2 weeks. In some embodiments, the dose is 0.4 mg/kg and is administered once about every 3 weeks. In some embodiments, the dose is 0.4 mg/kg and is administered once about every 4 weeks. In some embodiments, the dose is 0.45 mg/kg and is administered once about every 1 week. In some embodiments, the dose is 0.45 mg/kg and is administered once about every 2 weeks. In some embodiments, the dose is 0.45 mg/kg and is administered once about every 3 weeks. In some embodiments, the dose is 0.45 mg/kg and is administered once about every 4 weeks. In some embodiments, the dose is 0.5 mg/kg and is administered once about every 1 week. In some embodiments, the dose is 0.5 mg/kg and is administered once about every 2 weeks. In some embodiments, the dose is 0.5 mg/kg and is administered once about every 3 weeks. In some embodiments, the dose is 0.5 mg/kg and is administered once about every 4 weeks. In some embodiments, the dose is 0.55 mg/kg and is administered once about every 1 week. In some embodiments, the dose is 0.55 mg/kg and is administered once about every 2 weeks. In some embodiments, the dose is 0.55 mg/kg and is administered once about every 3 weeks. In some embodiments, the dose is 0.55 mg/kg and is administered once about every 4 weeks. In some embodiments, the dose is 0.6 mg/kg and is administered once about every 1 week. In some embodiments, the dose is 0.6 mg/kg and is administered once about every 2 weeks. In some embodiments, the dose is 0.6 mg/kg and is administered once about every 3 weeks. In some embodiments, the dose is 0.6 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered once about every 3 weeks. In some embodiments, the dose is 0.7 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered once about every 3 weeks. In some embodiments, the dose is 0.8 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 0.95 mg/kg and is administered once about every 1 week. In some embodiments, the dose is 0.95 mg/kg and is administered once about every 2 weeks. In some embodiments, the dose is 0.95 mg/kg and is administered once about every 3 weeks. In some embodiments, the dose is 0.95 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.05 mg/kg and is administered once about every 1 week. In some embodiments, the dose is 1.05 mg/kg and is administered once about every 2 weeks. In some embodiments, the dose is 1.05 mg/kg and is administered once about every 3 weeks. In some embodiments, the dose is 1.05 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.15 mg/kg and is administered once about every 1 week. In some embodiments, the dose is 1.15 mg/kg and is administered once about every 2 weeks. In some embodiments, the dose is 1.15 mg/kg and is administered once about every 3 weeks. In some embodiments, the dose is 1.15 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.25 mg/kg and is administered once about every 1 week. In some embodiments, the dose is 1.25 mg/kg and is administered once about every 2 weeks. In some embodiments, the dose is 1.25 mg/kg and is administered once about every 3 weeks. In some embodiments, the dose is 1.25 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.35 mg/kg and is administered once about every 1 week. In some embodiments, the dose is 1.35 mg/kg and is administered once about every 2 weeks. In some embodiments, the dose is 1.35 mg/kg and is administered once about every 3 weeks. In some embodiments, the dose is 1.35 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.45 mg/kg and is administered once about every 1 week. In some embodiments, the dose is 1.45 mg/kg and is administered once about every 2 weeks. In some embodiments, the dose is 1.45 mg/kg and is administered once about every 3 weeks. In some embodiments, the dose is 1.45 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.55 mg/kg and is administered once about every 1 week. In some embodiments, the dose is 1.55 mg/kg and is administered once about every 2 weeks. In some embodiments, the dose is 1.55 mg/kg and is administered once about every 3 weeks. In some embodiments, the dose is 1.55 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.65 mg/kg and is administered once about every 1 week. In some embodiments, the dose is 1.65 mg/kg and is administered once about every 2 weeks. In some embodiments, the dose is 1.65 mg/kg and is administered once about every 3 weeks. In some embodiments, the dose is 1.65 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.75 mg/kg and is administered once about every 1 week. In some embodiments, the dose is 1.75 mg/kg and is administered once about every 2 weeks. In some embodiments, the dose is 1.75 mg/kg and is administered once about every 3 weeks. In some embodiments, the dose is 1.75 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.2 mg/kg and is administered once about every 1 week. In some embodiments, the dose is 2.2 mg/kg and is administered once about every 2 weeks. In some embodiments, the dose is 2.2 mg/kg and is administered once about every 3 weeks. In some embodiments, the dose is 2.2 mg/kg and is administered once about every 4 weeks. In some embodiments, the dose is 2.3 mg/kg and is administered once about every 1 week. In some embodiments, the dose is 2.3 mg/kg and is administered once about every 2 weeks. In some embodiments, the dose is 2.3 mg/kg and is administered once about every 3 weeks. In some embodiments, the dose is 2.3 mg/kg and is administered once about every 4 weeks. In some embodiments, the dose is 2.4 mg/kg and is administered once about every 1 week. In some embodiments, the dose is 2.4 mg/kg and is administered once about every 2 weeks. In some embodiments, the dose is 2.4 mg/kg and is administered once about every 3 weeks. In some embodiments, the dose is 2.4 mg/kg and is administered once about every 4 weeks. In some embodiments, the dose is 2.5 mg/kg and is administered once about every 1 week. In some embodiments, the dose is 2.5 mg/kg and is administered once about every 2 weeks. In some embodiments, the dose is 2.5 mg/kg and is administered once about every 3 weeks. In some embodiments, the dose is 2.5 mg/kg and is administered once about every 4 weeks. In some embodiments, the dose is 2.6 mg/kg and is administered once about every 1 week. In some embodiments, the dose is 2.7 mg/kg and is administered once about every 2 weeks. In some embodiments, the dose is 2.7 mg/kg and is administered once about every 3 weeks. In some embodiments, the dose is 2.7 mg/kg and is administered once about every 4 weeks. In some embodiments, the dose is 2.8 mg/kg and is administered once about every 1 week. In some embodiments, the dose is 2.8 mg/kg and is administered once about every 2 weeks. In some embodiments, the dose is 2.8 mg/kg and is administered once about every 3 weeks. In some embodiments, the dose is 2.8 mg/kg and is administered once about every 4 weeks. In some embodiments, the dose is 2.9 mg/kg and is administered once about every 1 week. In some embodiments, the dose is 2.9 mg/kg and is administered once about every 2 weeks. In some embodiments, the dose is 2.9 mg/kg and is administered once about every 3 weeks. In some embodiments, the dose is 2.9 mg/kg and is administered once about every 4 weeks. In some embodiments, the dose is 3.0 mg/kg and is administered once about every 1 week. In some embodiments, the dose is 3.0 mg/kg and is administered once about every 2 weeks. In some embodiments, the dose is 3.0 mg/kg and is administered once about every 3 weeks. In some embodiments, the dose is 3.0 mg/kg and is administered once about every 4 weeks. In some embodiments, the dose is 3.1 mg/kg and is administered once about every 1 week. In some embodiments, the dose is 3.1 mg/kg and is administered once about every 2 weeks. In some embodiments, the dose is 3.1 mg/kg and is administered once about every 3 weeks. In some embodiments, the dose is 3.1 mg/kg and is administered once about every 4 weeks. In some embodiments, the dose is 3.2 mg/kg and is administered once about every 1 week. In some embodiments, the dose is 3.2 mg/kg and is administered once about every 2 weeks. In some embodiments, the dose is 3.2 mg/kg and is administered once about every 3 weeks. In some embodiments, the dose is 3.2 mg/kg and is administered once about every 4 weeks. In some embodiments, the dose is 3.3 mg/kg and is administered once about every 1 week. In some embodiments, the dose is 3.3 mg/kg and is administered once about every 2 weeks. In some embodiments, the dose is 3.3 mg/kg and is administered once about every 3 weeks. In some embodiments, the dose is 3.3 mg/kg and is administered once about every 4 weeks. In some embodiments, the dose is 3.4 mg/kg and is administered once about every 1 week. In some embodiments, the dose is 3.4 mg/kg and is administered once about every 2 weeks. In some embodiments, the dose is 3.4 mg/kg and is administered once about every 3 weeks. In some embodiments, the dose is 3.4 mg/kg and is administered once about every 4 weeks. In some embodiments, the dose is 3.5 mg/kg and is administered once about every 1 week. In some embodiments, the dose is 3.5 mg/kg and is administered once about every 2 weeks. In some embodiments, the dose is 3.5 mg/kg and is administered once about every 3 weeks. In some embodiments, the dose is 3.5 mg/kg and is administered once about every 4 weeks. In some embodiments, the dose is 3.6 mg/kg and is administered once about every 1 week. In some embodiments, the dose is 3.7 mg/kg and is administered once about every 2 weeks. In some embodiments, the dose is 3.7 mg/kg and is administered once about every 3 weeks. In some embodiments, the dose is 3.7 mg/kg and is administered once about every 4 weeks. In some embodiments, the dose is 3.8 mg/kg and is administered once about every 1 week. In some embodiments, the dose is 3.8 mg/kg and is administered once about every 2 weeks. In some embodiments, the dose is 3.8 mg/kg and is administered once about every 3 weeks. In some embodiments, the dose is 3.8 mg/kg and is administered once about every 4 weeks. In some embodiments, the dose is 3.9 mg/kg and is administered once about every 1 week. In some embodiments, the dose is 3.9 mg/kg and is administered once about every 2 weeks. In some embodiments, the dose is 3.9 mg/kg and is administered once about every 3 weeks. In some embodiments, the dose is 3.9 mg/kg and is administered once about every 4 weeks. In some embodiments, the dose is 4.0 mg/kg and is administered once about every 1 week. In some embodiments, the dose is 4.0 mg/kg and is administered once about every 2 weeks. In some embodiments, the dose is 4.0 mg/kg and is administered once about every 3 weeks. In some embodiments, the dose is 4.0 mg/kg and is administered once about every 4 weeks. In some embodiments, the dose is 4.1 mg/kg and is administered once about every 1 week. In some embodiments, the dose is 4.1 mg/kg and is administered once about every 2 weeks. In some embodiments, the dose is 4.1 mg/kg and is administered once about every 3 weeks. In some embodiments, the dose is 4.1 mg/kg and is administered once about every 4 weeks. In some embodiments, the dose is 4.2 mg/kg and is administered once about every 1 week. In some embodiments, the dose is 4.2 mg/kg and is administered once about every 2 weeks. In some embodiments, the dose is 4.2 mg/kg and is administered once about every 3 weeks. In some embodiments, the dose is 4.2 mg/kg and is administered once about every 4 weeks. In some embodiments, the dose is 4.3 mg/kg and is administered once about every 1 week. In some embodiments, the dose is 4.3 mg/kg and is administered once about every 2 weeks. In some embodiments, the dose is 4.3 mg/kg and is administered once about every 3 weeks. In some embodiments, the dose is 4.3 mg/kg and is administered once about every 4 weeks. In some embodiments, the dose is 4.4 mg/kg and is administered once about every 1 week. In some embodiments, the dose is 4.4 mg/kg and is administered once about every 2 weeks. In some embodiments, the dose is 4.4 mg/kg and is administered once about every 3 weeks. In some embodiments, the dose is 4.4 mg/kg and is administered once about every 4 weeks. In some embodiments, the dose is 4.5 mg/kg and is administered once about every 1 week. In some embodiments, the dose is 4.5 mg/kg and is administered once about every 2 weeks. In some embodiments, the dose is 4.5 mg/kg and is administered once about every 3 weeks. In some embodiments, the dose is 4.5 mg/kg and is administered once about every 4 weeks. In some embodiments, the dose is 4.6 mg/kg and is administered once about every 1 week. In some embodiments, the dose is 4.7 mg/kg and is administered once about every 2 weeks. In some embodiments, the dose is 4.7 mg/kg and is administered once about every 3 weeks. In some embodiments, the dose is 4.7 mg/kg and is administered once about every 4 weeks. In some embodiments, the dose is 4.8 mg/kg and is administered once about every 1 week. In some embodiments, the dose is 4.8 mg/kg and is administered once about every 2 weeks. In some embodiments, the dose is 4.8 mg/kg and is administered once about every 3 weeks. In some embodiments, the dose is 4.8 mg/kg and is administered once about every 4 weeks. In some embodiments, the dose is 4.9 mg/kg and is administered once about every 1 week. In some embodiments, the dose is 4.9 mg/kg and is administered once about every 2 weeks. In some embodiments, the dose is 4.9 mg/kg and is administered once about every 3 weeks. In some embodiments, the dose is 4.9 mg/kg and is administered once about every 4 weeks. In some embodiments, the dose is 5.0 mg/kg and is administered once about every 1 week. In some embodiments, the dose is 5.0 mg/kg and is administered once about every 2 weeks. In some embodiments, the dose is 5.0 mg/kg and is administered once about every 3 weeks. In some embodiments, the dose is 5.0 mg/kg and is administered once about every 4 weeks. In some embodiments, the dose is 0.1 mg/kg and is administered once every 3 weeks. In some embodiments, the dose is 0.1 mg/kg and is administered once every 3 weeks and the antibody-drug conjugate is SGN-CD30C. In some embodiments, the dose is 0.5 mg/kg and is administered once every 3 weeks. In some embodiments, the dose is 0.5 mg/kg and is administered once every 3 weeks and the antibody-drug conjugate is SGN-CD30C. In some embodiments, the dose is 0.1 mg/kg and is administered on about day 1 of about a 21-day treatment cycle and the antibody-drug conjugate is SGN-CD30C. In some embodiments, the dose is 0.1 mg/kg and is administered on day 1 of a 21-day treatment cycle and the antibody-drug conjugate is SGN-CD30C. In some embodiments, the dose is 0.5 mg/kg and is administered on about day 1 of about a 21-day treatment cycle and the antibody-drug conjugate is SGN-CD30C. In some embodiments, the dose is 0.5 mg/kg and is administered on day 1 of a 21-day treatment cycle and the antibody-drug conjugate is SGN-CD30C. The present invention encompasses embodiments wherein the subject remains on the 21-day treatment cycle for at least 2, 3, 4, 5, 6, 7, 8, 9, 10, 11, 12 or more cycles. In another embodiment, the subject remains on the 21-day treatment cycle for between 2 and 48 cycles, such as between 2 and 36 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles. In some embodiments, the subject remains on the 21-day treatment cycle for 12 cycles or more, such as 16 cycles or more, such as 24 cycles or more, such as 36 cycles or more. In some embodiments, the 21-day treatment cycle is administered for no more than 3, no more than 4, no more than 5, or no more than 6 four-week treatment cycles. The number of treatment cycles suitable for any specific subject or group of subjects may be determined by a person of skill in the art, typically a physician. In some embodiments, for a subject weighing more than 100 kg, the dose of the anti-CD30 antibody-drug conjugate administered is the amount that would be administered if the subject weighed 100 kg. In some embodiments, for a subject weighing more than 100 kg, the dose of the anti-CD30 antibody-drug conjugate administered is 10 mg. In some embodiments, for a subject weighing more than 100 kg, the dose of the anti-CD30 antibody-drug conjugate administered is 50 mg.

In some embodiments, the dose of the anti-CD30 antibody-drug conjugate described herein is about 0.05 mg/kg to about 5 mg/kg and is administered about every 1 to 4 weeks. In some embodiments, the dose of the anti-CD30 antibody-drug conjugate described herein is about 0.05 mg/kg to about 5 mg/kg and is administered about every 1 to 4 weeks and the antibody-drug conjugate is SGN-CD30C. In some embodiments, the anti-CD30 antibody-drug conjugate described herein is administered by intravenous infusion.

In some embodiments, the dose of the anti-CD30 antibody-drug conjugate described herein is about 0.1 mg/kg to about 3.2 mg/kg and is administered about every 1 to 4 weeks. In some embodiments, the dose of the anti-CD30 antibody-drug conjugate described herein is about 0.1 mg/kg to about 3.2 mg/kg and is administered about every 1 to 4 weeks and the antibody-drug conjugate is SGN-CD30C. In some embodiments, the anti-CD30 antibody-drug conjugate described herein is administered by intravenous infusion.

In some embodiments, the dose of the anti-CD30 antibody-drug conjugate described herein is about 0.1 mg/kg to about 2.7 mg/kg and is administered about every 1 to 4 weeks. In some embodiments, the dose of the anti-CD30 antibody-drug conjugate described herein is about 0.1 mg/kg to about 2.7 mg/kg and is administered about every 1 to 4 weeks and the antibody-drug conjugate is SGN-CD30C. In some embodiments, the anti-CD30 antibody-drug conjugate described herein is administered by intravenous infusion.

In some embodiments, the dose of the anti-CD30 antibody-drug conjugate described herein is 0.1 mg/kg and is administered once about every 3 weeks (e.g., +3 days). In some embodiments, the dose of the anti-CD30 antibody-drug conjugate described herein is 0.1 mg/kg and is administered once every 3 weeks. In some embodiments, the dose of the anti-CD30 antibody-drug conjugate is 0.1 mg/kg and is administered once every 3 weeks and the antibody-drug conjugate is SGN-CD30C. In some embodiments, the anti-CD30 antibody-drug conjugate described herein is administered by intravenous infusion.

In some embodiments, the dose of the anti-CD30 antibody-drug conjugate described herein is 0.5 mg/kg and is administered once about every 3 weeks (e.g., +3 days). In some embodiments, the dose of the anti-CD30 antibody-drug conjugate described herein is 0.5 mg/kg and is administered once every 3 weeks. In some embodiments, the dose of the anti-CD30 antibody-drug conjugate is 0.5 mg/kg and is administered once every 3 weeks and the antibody-drug conjugate is SGN-CD30C. In some embodiments, the anti-CD30 antibody-drug conjugate described herein is administered by intravenous infusion.

In some embodiments, the dose of the anti-CD30 antibody-drug conjugate described herein is 0.1 mg/kg and is administered on day 1 of about a 21-day (e.g., +3 days) treatment cycle. In some embodiments, the dose of the anti-CD30 antibody-drug conjugate described herein is 0.1 mg/kg and is administered on day 1 of a 21-day treatment cycle. In some embodiments, the dose of the anti-CD30 antibody-drug conjugate described herein is 0.1 mg/kg and is administered on day 1 of a 21-day treatment cycle. and the antibody-drug conjugate is SGN-CD30C. In some embodiments, the anti-CD30 antibody-drug conjugate described herein is administered by intravenous infusion.

In some embodiments, the dose of the anti-CD30 antibody-drug conjugate described herein is 0.5 mg/kg and is administered on day 1 of about a 21-day (e.g., +3 days) treatment cycle. In some embodiments, the dose of the anti-CD30 antibody-drug conjugate described herein is 0.5 mg/kg and is administered on day 1 of a 21-day treatment cycle. In some embodiments, the dose of the anti-CD30 antibody-drug conjugate described herein is 0.5 mg/kg and is administered on day 1 of a 21-day treatment cycle. and the antibody-drug conjugate is SGN-CD30C. In some embodiments, the anti-CD30 antibody-drug conjugate described herein is administered by intravenous infusion.

E. Treatment Outcome

In some embodiments, the invention provides a method of modulating the immune response in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. The invention also provides a method of decreasing the number of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the number of CD30⁺ Treg cells is decreased relative to the number of CD30⁺ Treg cells in the subject prior to administration of the antibody-drug conjugate. In some embodiments, the number of CD30+ Treg cells is decreased at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 75%, at least about 80%, at least about 85%, at least about 90%, or at least about 95%. The invention also provides a method of decreasing the activity of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof. In some embodiments, the decrease in the activity of CD30⁺ Treg cells is relative to the activity of CD30⁺ Treg cells in the subject prior to administration of the antibody-drug conjugate. In some embodiments, the activity of CD30+ Treg cells is decreased at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 75%, at least about 80%, at least about 85%, at least about 90%, or at least about 95%. In some embodiments, the CD30+ Treg cells are inducible T regulatory (iTreg) cells. In some embodiments, the CD30+ Treg cells are peripheral blood T regulatory (pbTreg) cells. In some embodiments, the anti-CD30 antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6. In some embodiments, the anti-CD30 antibody or antigen-binding fragment thereof of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8. In some embodiments, the anti-CD30 antibody or antigen-binding fragment thereof of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8. In some embodiments, the anti-CD30 antibody or antigen-binding fragment thereof of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8. In some embodiments, the anti-CD30 antibody or antigen-binding fragment thereof of the antibody-drug conjugate comprises a heavy chain variable region comprising the amino acid sequence of SEQ ID NO: 7 and a light chain variable region comprising the amino acid sequence of SEQ ID NO: 8. In some embodiments, the anti-CD30 antibody or antigen-binding fragment thereof of the antibody-drug conjugate is cAC10. In some embodiments, the antibody-drug conjugate is SGN-CD30C.

In some embodiments, the subject has cancer. In some embodiments, administering an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein results in an improvement in one or more therapeutic effects in the subject after administration of the antibody-drug conjugate relative to a baseline.

In some embodiments, the one or more therapeutic effects is the objective response rate, the duration of response, the time to response, progression free survival, overall survival, or any combination thereof. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment is assessed using the Lugano Classification Revised Staging System for nodal non-Hodgkin and Hodgkin lymphomas as described in Cheson B D, et al. J Clin Oncol. 32(27):3059-68 (2014). In some embodiments, the criteria for response assessment is as described in the following table:

Revised Criteria For Response Assessment

| Response and Site | PET-CT-Based Response | CT-Based Response |
|---|---|---|
| Complete | Complete metabolic response | Complete radiologic response (all of the |
| | | following) |
| Lymph nodes and | Score 1, 2, or 3″ with or without a residual | Target nodes/nodal masses must regress to |
| extralymphatic | mass on 5PS+ | ≤1.5 cm in LDi |
| sites | It is recognized that in Waldeyer's ring or | No extralymphatic sites of disease |
| | extranodal sites with high physiologic | |
| | uptake or with activation within spleen | |
| | or marrow log, with chemotherapy or | |
| | myeloid colony-stimulating factors), | |
| | update may be greater than normal | |
| | mediastinum and/or liver. In this | |
| | circumstance, complete metabolic | |
| | response may be inferred if update at | |
| | sites of initial involvement is no greater | |
| | than surrounding normal tissue even if | |
| | the tissue has high physiologic uptake | |
| Nonmeasured | Not applicable | Absent |
| lesion | | |
| Organ | Not applicable | Regress to normal |
| enlargement | | |
| New lesions | None | None |
| Bone marrow | No evidence of FDG-avid disease in | Normal by morphology; if indeterminate, |
| | marrow | IHC negative |
| Partial | Partial metabolic response | Partial remission (all of the following) |
| Lymph nodes and | Score 4 or 5+ with reduced uptake | ≥50% decrease in SPD of up to 6 target |
| extralymphatic | compared with baseline and residual | measurable nodes and extranodal sites |
| sites | mass(es) of any size | |
| | At interim, these findings suggest | When a lesion is too small to measure on |
| | responding disease | CT, assign 5 mm × 5 mm as the default |
| | | value |
| | At end of treatment, these findings | When no longer visible, 0 × 0 mm |
| | indicate residual disease | For a node >5 mm × 5 mm, but smaller |
| | | than normal, use actual measurement for |
| | | calculation |
| Nonmeasured | Not applicable | Absent/normal, regressed, but no increase |
| lesion | | |
| Organ | Not applicable | Spleen must have regressed by >50% in |
| enlargement | | length beyond normal |
| New lesions | None | None |
| Bone marrow | Residual uptake higher than uptake in | Not applicable |
| | normal marrow but reduced compared | |
| | with baseline (diffuse uptake | |
| | compatible with reactive changes from | |
| | chemotherapy allowed). If there are | |
| | persistent focal changes in the marrow | |
| | in the context of a nodal response, | |
| | consideration should be given to further | |
| | evaluation with MRI or biopsy or an | |
| | interval scan | |
| No response or | No metabolic response | Stable disease |
| stable disease | | |
| Target | Score 4 or 5 with no significant change in | <50% decrease from baseline in SPD of up |
| nodes/nodal | FDG uptake from baseline at interim or | to 6 dominant, measurable nodes and |
| masses, | end of treatment | extranodal sites; no criteria for |
| extranodal | | progressive disease are met |
| lesions | | |
| Nonmeasured | Not applicable | No increase consistent with progression |
| lesion | | |
| Organ | Not applicable | No increase consistent with progression |
| enlargement | | |
| New lesions | None | None |
| Bone marrow | No change from baseline | Not applicable |
| Progressive disease | Progressive metabolic disease | Progressive disease requires at least 1 of the |
| | | following |
| Individual target | Score 4 or 5 with an increase in intensity | PPD progression: |
| nodes/nodal | of uptake from baseline and/or | |
| masses | | |
| Extranodal lesions | New FDG-avid foci consistent with | An individual node/lesion must be |
| | lymphoma at interim or end-of- | abnormal with: |
| | treatment assessment | LDi >1.5 cm and |
| | | Increase by ≥50% from PPD nadir and |
| | | An increase in LDi or SDi from nadir |
| | | 0.5 cm for lesions ≤2 cm |
| | | 1.0 cm for lesions >2 cm |
| | | In the setting of splenomegaly, the splenic |
| | | length must increase by >50% of the |
| | | extent of its prior increase beyond |
| | | baseline (eg. a 15-cm spleen must |
| | | increase to >16 cm). If no prior |
| | | splenomegaly, must increase by at least 2 |
| | | cm from baseline |
| | | New or recurrent splenomegaly |
| Nonmeasured | None | New or clear progression of preexisting |
| lesions | | nonmeasured lesions |
| New lesions | New FDG-avid foci consistent with | Regrowth of previously resolved lesions |
| | lymphoma rather than another etiology | A new node >1.5 cm in any axis |
| | (eg. infection, inflammation). If | |
| | uncertain regarding etiology of new | |
| | lesions, biopsy or interval scan may be | A new extranodal site >1.0 cm in any axis; |
| | considered | if <1.0 cm in any axis, its presence must |
| | | be unequivocal and must be attributable |
| | | to lymphoma |
| | | Assessable disease of any size |
| | | unequivocally attributable to lymphoma |
| Bone marrow | New or recurrent FDG-avid foci | New or recurrent involvement |

Abbreviations:
5PS, 5-point scale;
CT, computed tomography;
FDG, fluorodeoxyglucose;
IHC, immunohistochemistry;
LDi, longest transverse diameter of a lesion;
MRI, magnetic resonance imaging;
PET, positron emission tomography;
PPD, cross product of the LDi and perpendicular diameter;
SDi, shortest axis perpendicular to the LDi;
SPD, sum of the product of the perpendicular diameters for multiple lesions.
*A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Nonmeasured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{†}PET 5PS:
1, no uptake above background;
2, uptake ≤mediastinum;
3, uptake >mediastinum but ≤liver;
4, uptake moderately >liver;
5, uptake markedly higher than liver and/or new lesions;
X, new areas of uptake unlikely to be related to lymphoma.

In one embodiment of the methods or uses or product for uses provided herein, the effectiveness of treatment with an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is assessed by measuring the objective response rate. In some embodiments, the objective response rate is the proportion of patients with tumor size reduction of a predefined amount and for a minimum period of time. In some embodiments the objective response rate is based upon Cheson criteria. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses described herein, response to treatment with an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is assessed by measuring the time of progression free survival after administration of the anti-CD30 antibody-drug conjugate or antigen-binding fragment thereof as described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate or antigen-binding fragment thereof as described herein. In some embodiments, the subject exhibits progression-free survival of at least about 6 months after administration of the anti-CD30 antibody-drug conjugate or antigen-binding fragment thereof as described herein. In some embodiments, the subject exhibits progression-free survival of at least about one year after administration of the anti-CD30 antibody-drug conjugate or antigen-binding fragment thereof as described herein. In some embodiments, the subject exhibits progression-free survival of at least about two years after administration of the anti-CD30 antibody-drug conjugate or antigen-binding fragment thereof as described herein. In some embodiments, the subject exhibits progression-free survival of at least about three years after administration of the anti-CD30 antibody-drug conjugate or antigen-binding fragment thereof as described herein. In some embodiments, the subject exhibits progression-free survival of at least about four years after administration of the anti-CD30 antibody-drug conjugate or antigen-binding fragment thereof as described herein. In some embodiments, the subject exhibits progression-free survival of at least about five years after administration of the anti-CD30 antibody-drug conjugate or antigen-binding fragment thereof as described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-CD30 antibody-drug conjugate or antigen-binding fragment thereof as described herein. In some embodiments, the subject exhibits progression-free survival of at least 6 months after administration of the anti-CD30 antibody-drug conjugate or antigen-binding fragment thereof as described herein. In some embodiments, the subject exhibits progression-free survival of at least one year after administration of the anti-CD30 antibody-drug conjugate or antigen-binding fragment thereof as described herein. In some embodiments, the subject exhibits progression-free survival of at least two years after administration of the anti-CD30 antibody-drug conjugate or antigen-binding fragment thereof as described herein. In some embodiments, the subject exhibits progression-free survival of at least three years after administration of the anti-CD30 antibody-drug conjugate or antigen-binding fragment thereof as described herein. In some embodiments, the subject exhibits progression-free survival of at least four years after administration of the anti-CD30 antibody-drug conjugate or antigen-binding fragment thereof as described herein. In some embodiments, the subject exhibits progression-free survival of at least five years after administration of the anti-CD30 antibody-drug conjugate or antigen-binding fragment thereof as described herein.

In one embodiment of the methods or uses or product for uses described herein, response to treatment with an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is assessed by measuring the time of overall survival after administration of the anti-CD30 antibody-drug conjugate or antigen-binding fragment thereof as described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate or antigen-binding fragment thereof as described herein. In some embodiments, the subject exhibits overall survival of at least about 6 months after administration of the anti-CD30 antibody-drug conjugate or antigen-binding fragment thereof as described herein. In some embodiments, the subject exhibits overall survival of at least about one year after administration of the anti-CD30 antibody-drug conjugate or antigen-binding fragment thereof as described herein. In some embodiments, the subject exhibits overall survival of at least about two years after administration of the anti-CD30 antibody-drug conjugate or antigen-binding fragment thereof as described herein. In some embodiments, the subject exhibits overall survival of at least about three years after administration of the anti-CD30 antibody-drug conjugate or antigen-binding fragment thereof as described herein. In some embodiments, the subject exhibits overall survival of at least about four years after administration of the anti-CD30 antibody-drug conjugate or antigen-binding fragment thereof as described herein. In some embodiments, the subject exhibits overall survival of at least about five years after administration of the anti-CD30 antibody-drug conjugate or antigen-binding fragment thereof as described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the anti-CD30 antibody-drug conjugate or antigen-binding fragment thereof as described herein. In some embodiments, the subject exhibits overall survival of at least 6 months after administration of the anti-CD30 antibody-drug conjugate or antigen-binding fragment thereof as described herein. In some embodiments, the subject exhibits overall survival of at least one year after administration of the anti-CD30 antibody-drug conjugate or antigen-binding fragment thereof as described herein. In some embodiments, the subject exhibits overall survival of at least two years after administration of the anti-CD30 antibody-drug conjugate or antigen-binding fragment thereof as described herein. In some embodiments, the subject exhibits overall survival of at least three years after administration of the anti-CD30 antibody-drug conjugate or antigen-binding fragment thereof as described herein. In some embodiments, the subject exhibits overall survival of at least four years after administration of the anti-CD30 antibody-drug conjugate or antigen-binding fragment thereof as described herein. In some embodiments, the subject exhibits overall survival of at least five years after administration of the anti-CD30 antibody-drug conjugate or antigen-binding fragment thereof as described herein.

In one embodiment of the methods or uses or product for uses described herein, response to treatment with an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is assessed by measuring the duration of response to the anti-CD30 antibody-drug conjugate or antigen-binding fragment thereof as described herein after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about 6 months after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about one year after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about two years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about three years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about four years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least about five years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least 6 months after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least one year after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least two years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least three years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least four years after administration of the anti-CD30 antibody-drug conjugate or antigen-binding fragment thereof as described herein. In some embodiments, the duration of response to the anti-CD30 antibody-drug conjugate or antigen-binding fragment thereof as described herein is at least five years after administration of the anti-CD30 antibody-drug conjugate or antigen-binding fragment thereof as described herein.

In some embodiments of the methods or uses or product for uses described herein, administering an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein to a subject results in a depletion of cancer cells in the subject. In some embodiments, administering the anti-CD30 antibody-drug conjugate or antigen-binding fragment thereof as described herein results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the anti-CD30 antibody-drug conjugate or antigen-binding fragment thereof as described herein to the subject. In some embodiments, the cancer cells are depleted by at least about 5% compared to the amount of cancer cells before administering the anti-CD30 antibody-drug conjugate or antigen-binding fragment thereof as described herein to the subject. In some embodiments, the cancer cells are depleted by at least about 10% compared to the amount of cancer cells before administering the anti-CD30 antibody-drug conjugate or antigen-binding fragment thereof as described herein to the subject. In some embodiments, the cancer cells are depleted by at least about 20% compared to the amount of cancer cells before administering the anti-CD30 antibody-drug conjugate or antigen-binding fragment thereof as described herein to the subject. In some embodiments, the cancer cells are depleted by at least about 30% compared to the amount of cancer cells before administering the anti-CD30 antibody-drug conjugate or antigen-binding fragment thereof as described herein to the subject. In some embodiments, the cancer cells are depleted by at least about 40% compared to the amount of cancer cells before administering the anti-CD30 antibody-drug conjugate or antigen-binding fragment thereof as described herein to the subject. In some embodiments, the cancer cells are depleted by at least about 50% compared to the amount of cancer cells before administering the anti-CD30 antibody-drug conjugate or antigen-binding fragment thereof as described herein to the subject. In some embodiments, the cancer cells are depleted by at least about 60% compared to the amount of cancer cells before administering the anti-CD30 antibody-drug conjugate or antigen-binding fragment thereof as described herein to the subject. In some embodiments, the cancer cells are depleted by at least about 70% compared to the amount of cancer cells before administering the anti-CD30 antibody-drug conjugate or antigen-binding fragment thereof as described herein to the subject. In some embodiments, the cancer cells are depleted by at least about 80% compared to the amount of cancer cells before administering the anti-CD30 antibody-drug conjugate or antigen-binding fragment thereof as described herein to the subject. In some embodiments, the cancer cells are depleted by at least about 90% compared to the amount of cancer cells before administering the anti-CD30 antibody-drug conjugate or antigen-binding fragment thereof as described herein to the subject. In some embodiments, the cancer cells are depleted by at least about 95% compared to the amount of cancer cells before administering the anti-CD30 antibody-drug conjugate or antigen-binding fragment thereof as described herein to the subject. In some embodiments, the cancer cells are depleted by at least about 99% compared to the amount of cancer cells before administering the anti-CD30 antibody-drug conjugate or antigen-binding fragment thereof as described herein to the subject. In some embodiments, the cancer cells are depleted by about 100% compared to the amount of cancer cells before administering the anti-CD30 antibody-drug conjugate or antigen-binding fragment thereof as described herein to the subject. In some embodiments, administering the anti-CD30 antibody-drug conjugate or antigen-binding fragment thereof as described herein results in a depletion of cancer cells by at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least about 80%, at least about 90%, at least 95%, or 100% compared to the amount of cancer cells before administering the anti-CD30 antibody-drug conjugate or antigen-binding fragment thereof as described herein to the subject. In some embodiments, the cancer cells are depleted by at least 5% compared to the amount of cancer cells before administering the anti-CD30 antibody-drug conjugate or antigen-binding fragment thereof as described herein to the subject. In some embodiments, the cancer cells are depleted by at least 10% compared to the amount of cancer cells before administering the anti-CD30 antibody-drug conjugate or antigen-binding fragment thereof as described herein to the subject. In some embodiments, the cancer cells are depleted by at least 20% compared to the amount of cancer cells before administering the anti-CD30 antibody-drug conjugate or antigen-binding fragment thereof as described herein to the subject. In some embodiments, the cancer cells are depleted by at least 30% compared to the amount of cancer cells before administering the anti-CD30 antibody-drug conjugate or antigen-binding fragment thereof as described herein to the subject. In some embodiments, the cancer cells are depleted by at least 40% compared to the amount of cancer cells before administering the anti-CD30 antibody-drug conjugate or antigen-binding fragment thereof as described herein to the subject. In some embodiments, the cancer cells are depleted by at least 50% compared to the amount of cancer cells before administering the anti-CD30 antibody-drug conjugate or antigen-binding fragment thereof as described herein to the subject. In some embodiments, the cancer cells are depleted by at least 60% compared to the amount of cancer cells before administering the anti-CD30 antibody-drug conjugate or antigen-binding fragment thereof as described herein to the subject. In some embodiments, the cancer cells are depleted by at least 70% compared to the amount of cancer cells before administering the anti-CD30 antibody-drug conjugate or antigen-binding fragment thereof as described herein to the subject. In some embodiments, the cancer cells are depleted by at least 80% compared to the amount of cancer cells before administering the anti-CD30 antibody-drug conjugate or antigen-binding fragment thereof as described herein to the subject. In some embodiments, the cancer cells are depleted by at least 90% compared to the amount of cancer cells before administering the anti-CD30 antibody-drug conjugate or antigen-binding fragment thereof as described herein to the subject. In some embodiments, the cancer cells are depleted by at least 95% compared to the amount of cancer cells before administering the anti-CD30 antibody-drug conjugate or antigen-binding fragment thereof as described herein to the subject. In some embodiments, the cancer cells are depleted by at least 99% compared to the amount of cancer cells before administering the anti-CD30 antibody-drug conjugate or antigen-binding fragment thereof as described herein to the subject. In some embodiments, the cancer cells are depleted by 100% compared to the amount of cancer cells before administering the anti-CD30 antibody-drug conjugate or antigen-binding fragment thereof as described herein to the subject.

### III. Compositions

In some aspects, also provided herein are compositions (e.g., pharmaceutical compositions and therapeutic formulations) comprising any of the anti-CD30 antibody-drug conjugates or antigen-binding fragments thereof as described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wilkins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In some embodiments provided herein, a formulation comprising an anti-CD30 antibody-drug conjugate or antigen-binding fragment thereof as described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of an anti-CD30 antibody-drug conjugates or antigen-binding fragments thereof as described herein for use in a method of decreasing the number of CD30+T regulatory cells as described herein. The invention also provides compositions comprising a population of an anti-CD30 antibody-drug conjugates or antigen-binding fragments thereof as described herein for use in a method of decreasing the activity of CD30+T regulatory cells as described herein. In some embodiments, provided herein are compositions comprising a population of antibody-drug conjugates, wherein the antibody-drug conjugates comprise a linker attached to a camptothecin, wherein the antibody-drug conjugate has the following structure:

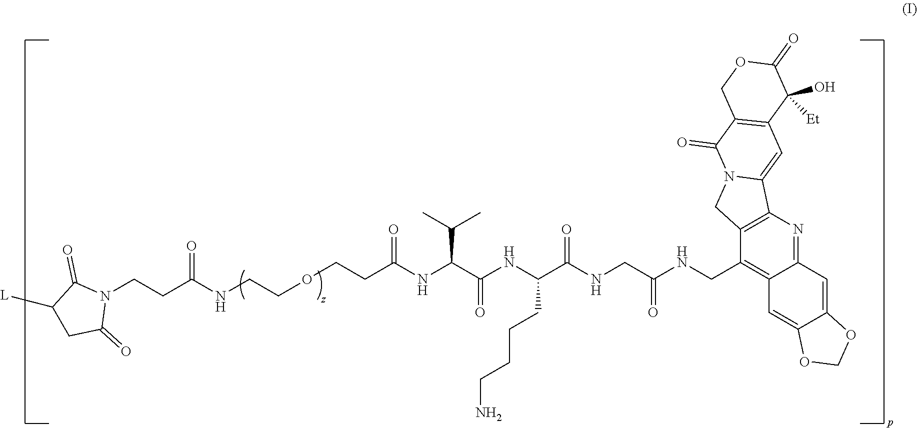

or a pharmaceutically acceptable salt thereof, wherein:
L is an antibody, such as the anti-CD30 antibody brentuximab,
z is an integer from 2 to 12, or is 2, 4, 8, or 12, and
p is 1-16, or is 2, 3, 4, 5, 6, 7, 8, 9, or 10, or is 2, 4 or 8. In some embodiments, p is an integer preferably ranging from 1 to 16, 1 to 12, 1 to 10, or 1 to 8. Individual camptothecin conjugate can be also be referred to as a camptothecin conjugate compound. In some embodiments, p is an integer from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, from 1 to 2, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, from 2 to 3, from 3 to 16, from 3 to 15, from 3 to 14, from 3 to 13, from 3 to 12, from 3 to 11, from 3 to 10, from 3 to 9, from 3 to 8, from 3 to 7, from 3 to 6, from 3 to 5, from 3 to 4, from 4 to 16, from 4 to 15, from 4 to 14, from 4 to 13, from 4 to 12, from 4 to 11, from 4 to 10, from 4 to 9, from 4 to 8, from 4 to 7, from 4 to 6, from 4 to 5, from 5 to 16, from 5 to 15, from 5 to 14, from 5 to 13, from 5 to 12, from 5 to 11, from 5 to 10, from 5 to 9, from 5 to 8, from 5 to 7, from 5 to 6, from 6 to 16, from 6 to 15, from 6 to 14, from 6 to 13, from 6 to 12, from 6 to 11, from 6 to 10, from 6 to 9, from 6 to 8, from 6 to 7, from 7 to 16, from 7 to 15, from 7 to 14, from 7 to 13, from 7 to 12, from 7 to 11, from 7 to 10, from 7 to 9, from 7 to 8, from 8 to 16, from 8 to 15, from 8 to 14, from 8 to 13, from 8 to 12, from 8 to 11, from 8 to 10, from 8 to 9, from 9 to 16, from 9 to 15, from 9 to 14, from 9 to 13, from 9 to 12, from 9 to 11, from 9 to 10, from 10 to 16, from 10 to 15, from 10 to 14, from 10 to 13, from 10 to 12, from 10 to 11, from 11 to 16, from 11 to 15, from 11 to 14, from 11 to 13, from 11 to 12, from 12 to 16, from 12 to 15, from 12 to 14, from 12 to 13, from 13 to 16, from 13 to 15, from 13 to 14, from 14 to 16, from 14 to 15, or from 15 to 16. In some embodiments, p is 1, 2, 3, 4, 5, 6, 7, 8, 9, 10, 11, 12, 13, 14, 15, or 16. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10. In some embodiments, p is 11. In some embodiments, p is 12. In some embodiments, p is 13. In some embodiments, p is 14. In some embodiments, p is 15. In some embodiments, p is 16. In some embodiments, the population is a mixed population of antibody-drug conjugates in which p varies from 1 to 16 for each antibody-drug conjugate. In some embodiments, the population is a homogenous population of antibody-drug conjugates with each antibody-drug conjugate having the same value for p.

In some embodiments, a composition comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is coadministered with one or more therapeutic agents to eliminate or reduce the severity of one or more adverse events. In some embodiments, a composition comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is coadministered with one or more therapeutic agents to prevent the development of the adverse event or to reduce the severity of the adverse event.

In some embodiments, a composition comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is coadministered with one or additional therapeutic agents. In some embodiments the coadministration is simultaneous or sequential. In some embodiments, the anti-CD30 antibody-drug conjugate described herein is administered simultaneously with the one or more additional therapeutic agents. In some embodiments, simultaneous means that the anti-CD30 antibody-drug conjugate described herein and the one or more therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and the one or more therapeutic agents are administered to the subject less than one hour apart, such as less than 30 minutes apart, less than 15 minutes apart, less than 10 minutes apart or less than 5 minutes apart. In some embodiments, the anti-CD30 antibody-drug conjugate described herein is administered sequentially with the one or more additional therapeutic agents. In some embodiments, sequential administration means that the anti-CD30 antibody-drug conjugate described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is coadministered with one or more therapeutic agents to eliminate or reduce the severity of one or more adverse events. In some embodiments the coadministration is simultaneous or sequential. In some embodiments, the anti-CD30 antibody-drug conjugate described herein is administered simultaneously with the one or more therapeutic agents to eliminate or reduce the severity of one or more adverse events. In some embodiments, simultaneous means that the anti-CD30 antibody-drug conjugate described herein and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-CD30 antibody-drug conjugate described herein is administered sequentially with the one or more therapeutic agents to eliminate or reduce the severity of one or more adverse events. In some embodiments, sequential administration means that the anti-CD30 antibody-drug conjugate described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-CD30 antibody-drug conjugate described herein is administered prior to the one or more therapeutic agents to eliminate or reduce the severity of one or more adverse events. In some embodiments, the one or more therapeutic agents to eliminate or reduce the severity of one or more adverse events is administered prior to the anti-CD30 antibody-drug conjugate described herein.

### IV. Articles of Manufacture and Kits

In another aspect, an article of manufacture or kit is provided which comprises an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein. The article of manufacture or kit may further comprise instructions for use of the antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein in the methods of the invention. Thus, in certain embodiments, the article of manufacture or kit comprises instructions for the use of the antibody-drug conjugate comprising an anti-CD30 antibody or an antigen-binding fragment thereof conjugated to a camptothecin or a functional analog thereof or a functional derivative thereof, as described herein in methods for decreasing the number of CD3⁰ T regulatory (Treg) cells in a subject comprising administering to the subject an effective amount of the antibody-drug conjugate comprising an anti-CD30 antibody or an antigen-binding fragment thereof conjugated to a camptothecin or a functional analog thereof or a functional derivative thereof, as described herein. In other embodiments, the article of manufacture or kit comprises instructions for the use of the antibody-drug conjugate comprising an anti-CD30 antibody or an antigen-binding fragment thereof conjugated to a camptothecin or a functional analog thereof or a functional derivative thereof, as described herein in methods for decreasing the activity of CD30 T regulatory (Treg) cells in a subject comprising administering to the subject an effective amount of the antibody-drug conjugate comprising an anti-CD30 antibody or an antigen-binding fragment thereof conjugated to a camptothecin or a functional analog thereof or a functional derivative thereof, as described herein. In some embodiments, the subject has cancer. In some embodiments, the cancer is a hematologic cancer. In some embodiments, the cancer is selected from the group consisting of Hodgkin lymphoma, non-Hodgkin lymphoma, anaplastic large cell lymphoma, peripheral T-cell lymphoma, or mycosis fungoides. In some embodiments, the cancer is Hodgkin lymphoma. In some embodiments, the Hodgkin lymphoma is classical Hodgkin lymphoma (cHL). In some embodiments, the cancer is non-Hodgkin lymphoma. In some embodiments, the non-Hodgkin lymphoma is diffuse large B-cell lymphoma (DLBCL). In some embodiments, the DLBCL is germinal-center B-cell like (GCB). In some embodiments, the DLBCL is non-GCB. In some embodiments, the cancer is anaplastic large cell lymphoma. In some embodiments, the anaplastic large cell lymphoma is systemic anaplastic large cell lymphoma. In some embodiments, the anaplastic large cell lymphoma is primary cutaneous anaplastic large cell lymphoma. In some embodiments, the non-Hodgkin lymphoma is a mature T-cell lymphoma. In some embodiments, the non-Hodgkin lymphoma is cutaneous T-cell lymphoma (CTCL). In some embodiments, the cancer is peripheral T-cell lymphoma. In some embodiments, the peripheral T-cell lymphoma is angioimmunoblastic T-cell lymphoma. In some embodiments, the cancer is mycosis fungoides. In some embodiments, the cancer is a non-hematologic cancer. In some embodiments, the non-hematologic cancer is a carcinoma. In some embodiments, the non-hematologic cancer is a sarcoma. In some embodiments, the non-hematologic cancer is a solid tumor. In some embodiments, the cancer is a CD30+ cancer, while in other embodiments, the cancer is a CD30− cancer. In some embodiments, the cancer is an advanced stage cancer. In some embodiments, the advanced stage cancer is a stage 3 or stage 4 cancer. In some embodiments, the advanced stage cancer is metastatic cancer. In some embodiments, the subject has been previously treated with one or more therapeutic agents and did not respond to the treatment. In some embodiments, the subject has been previously treated with one or more therapeutic agents and relapsed after the treatment. In some embodiments, the subject has been previously treated with one or more therapeutic agents and has experienced disease progression during treatment. In some embodiments, the subject has previously received allogenic stem cell transplant to treat the cancer. In some embodiments, the subject has previously received autologous stem cell transplant to treat the cancer. In some embodiments, the subject relapsed following stem cell transplant. In some embodiments, the subject has previously received CAR-T therapy. In some embodiments, the subject relapsed after CAR-T therapy. In some embodiments, the cancer is recurrent cancer. In some embodiments, the subject has not previously been treated for the cancer. In some embodiments, the subject has not been previously treated with an antibody-drug conjugate that binds to CD30. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cancer in a subject as described herein. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-CD30 antibody-drug conjugated to a camptothecin or a functional analog thereof or a functional derivative thereof is a first medicament, and which article or kit further comprises instructions on the label or package insert for treating the subject with the second medicament, in an effective amount. In some embodiments, the label or package insert indicates that the first and second medicament are to be administered sequentially or simultaneously, as described herein.

In some embodiments, the antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, as described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### ENUMERATED EMBODIMENTS

1. A method of decreasing the number of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof.

2. The method of embodiment 1, wherein the number of CD30 Treg cells is decreased relative to the number of CD30⁺ Treg cells in the subject prior to administration of the antibody-drug conjugate.

3. A method of decreasing the activity of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof.

4. The method of embodiment 3, wherein the decrease in the activity of CD30 Treg cells is relative to the activity of CD30 Treg cells in the subject prior to administration of the antibody-drug conjugate.

5. The method of any one of embodiments 1-4, wherein the CD30+ Treg cells are inducible T regulatory (iTreg) cells.

6. The method of any one of embodiments 1-5, wherein the CD30+ Treg cells are peripheral blood T regulatory (pbTreg) cells.

7. The method of any one of embodiments 1-6, wherein the anti-CD30 antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

8. The method of any one of embodiments 1-7, wherein the anti-CD30 antibody or antigen-binding fragment thereof of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8.

9. The method of any one of embodiments 1-7, wherein the anti-CD30 antibody or antigen-binding fragment thereof of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8.

10. The method of any one of embodiments 1-7, wherein the anti-CD30 antibody or antigen-binding fragment thereof of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8.

11. The method of any one of embodiments 1-6, wherein the anti-CD30 antibody or antigen-binding fragment thereof of the antibody-drug conjugate comprises a heavy chain variable region comprising the amino acid sequence of SEQ ID NO: 7 and a light chain variable region comprising the amino acid sequence of SEQ ID NO: 8.

12. The method of any one of embodiments 1-6, wherein the anti-CD30 antibody or antigen-binding fragment thereof of the antibody-drug conjugate is cAC10.

13. The method of any one of embodiments 1-12, wherein the antibody-drug conjugate comprises an anti-CD30 antibody or an antigen-binding fragment thereof conjugated to a camptothecin or a functional analog thereof or a functional derivative thereof forming a camptothecin conjugate of Formula (IC):

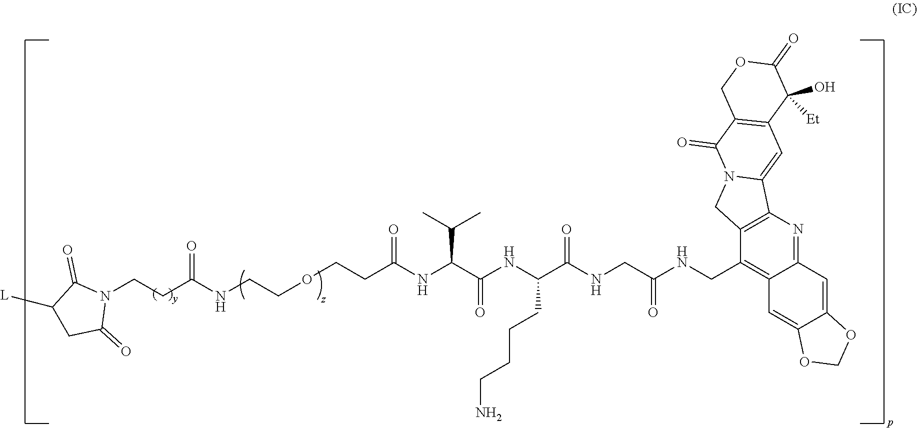

or a pharmaceutically acceptable salt thereof, wherein:
L is the anti-CD30 antibody or an antigen-binding fragment thereof,
y is 1, 2, 3, or 4, or is 1 or 4,
z is an integer from 2 to 12, or is 2, 4, 8, or 12, and
p is 1-16, or is 2, 3, 4, 5, 6, 7, 8, 9, or 10, or is 2, 4 or 8.

14. The method of embodiment 13, wherein y is 1.

15. The method of embodiment 13 or embodiment 14, wherein z is 8.

16. The method of any one of embodiments 13-15, wherein p is 8.

17. The method of any one of embodiments 1-16, wherein the antibody-drug conjugate is SGN-CD30C.

18. The method of any one of embodiments 1-17, wherein the antibody-drug conjugate is administered at a dose of 0.01 mg/kg to 5 mg/kg of the subject's bodyweight.

19. The method of embodiment 18, wherein the antibody-drug conjugate is administered at a dose of 0.1 mg/kg to 2 mg/kg of the subject's bodyweight.

20. The method of embodiment 18, wherein the antibody-drug conjugate is administered at a dose of about 0.1 mg/kg of the subject's bodyweight.

21. The method of embodiment 18, wherein the antibody-drug conjugate is administered at a dose of about 0.5 mg/kg of the subject's bodyweight.

22. The method of any one of embodiments 1-21, wherein the antibody-drug conjugate is administered to the subject once about every 3 weeks.

23. The method of any one of embodiments 1-21, wherein the antibody-drug conjugate is administered to the subject once every 3 weeks.

24. The method of any one of embodiments 1-22, wherein the antibody-drug conjugate is administered to the subject on about day 1 of about a 21-day treatment cycle.

25. The method of any one of embodiments 1-22, wherein the antibody-drug conjugate is administered to the subject on day 1 of a 21-day treatment cycle.

26. The method of any one of embodiments 1-25, wherein the antibody-drug conjugate is administered by intravenous infusion.

27. The method of any one of embodiments 1-26 further comprising the administration of granulocyte-colony stimulating factor (G-CSF) to the subject.

28. The method of embodiment 27, wherein the G-CSF is administered 1 to 3 days after the administration of the antibody-drug conjugate.

29. The method of embodiment 27 or embodiment 28, wherein the G-CSF is selected from the group consisting of filgrastim, PEG-filgrastim, lenograstim, and tbo-filgrastim.

30. The method of any one of embodiments 1-29, wherein the method further comprises administering one or more additional therapeutic agents capable of modulating the immune response.

31. The method of any one of embodiments 1-30, wherein the subject has cancer.

32. The method of embodiment 31, wherein the cancer is a hematologic cancer.

33. The method of embodiment 31 or embodiment 32, wherein the cancer is selected from the group consisting of Hodgkin lymphoma, non-Hodgkin lymphoma, anaplastic large cell lymphoma, peripheral T-cell lymphoma, or mycosis fungoides.

34. The method of embodiment 33, wherein the cancer is Hodgkin lymphoma.

35. The method of embodiment 34, wherein the Hodgkin lymphoma is classical Hodgkin lymphoma (cHL).

36. The method of embodiment 33, wherein the cancer is non-Hodgkin lymphoma.

37. The method of embodiment 36, wherein the non-Hodgkin lymphoma is diffuse large B-cell lymphoma (DLBCL).

38. The method of embodiment 37, wherein the DLBCL is germinal-center B-cell like (GCB).

39. The method of embodiment 37, wherein the DLBCL is non-GCB.

40. The method of embodiment 33, wherein the cancer is anaplastic large cell lymphoma.

41. The method of embodiment 40, wherein the anaplastic large cell lymphoma is systemic anaplastic large cell lymphoma.

42. The method of embodiment 40, wherein the anaplastic large cell lymphoma is primary cutaneous anaplastic large cell lymphoma.

43. The method of embodiment 36, wherein the non-Hodgkin lymphoma is a mature T-cell lymphoma.

44. The method of embodiment 36, wherein the non-Hodgkin lymphoma is cutaneous T-cell lymphoma (CTCL).

45. The method of embodiment 33, wherein the cancer is peripheral T-cell lymphoma.

46. The method of embodiment 45, wherein the peripheral T-cell lymphoma is angioimmunoblastic T-cell lymphoma.

47. The method of embodiment 33, wherein the cancer is mycosis fungoides.

48. The method of embodiment 31, wherein the cancer is a non-hematologic cancer.

49. The method of embodiment 48, wherein the non-hematologic cancer is a carcinoma.

50. The method of embodiment 48, wherein the non-hematologic cancer is a sarcoma.

51. The method of embodiment 48, wherein the non-hematologic cancer is a solid tumor.

52. The method of any one of embodiments 31-51, wherein the cancer is an advanced stage cancer.

53. The method of embodiment 52, wherein the advanced stage cancer is a stage 3 or stage 4 cancer.

54. The method of embodiment 52 or embodiment 53, wherein the advanced stage cancer is metastatic cancer.

55. The method of any one of embodiments 31-54, wherein the subject has been previously treated with one or more therapeutic agents and did not respond to the treatment.

56. The method of any one of embodiments 31-54, wherein the subject has been previously treated with one or more therapeutic agents and relapsed after the treatment.

57. The method of any one of embodiments 31-54, wherein the subject has been previously treated with one or more therapeutic agents and has experienced disease progression during treatment.

58. The method of any one of embodiments 31-57, wherein the subject has previously received allogenic stem cell transplant to treat the cancer.

59. The method of any one of embodiments 31-57, wherein the subject has previously received autologous stem cell transplant to treat the cancer.

60. The method of embodiment 58 or embodiment 59, wherein the subject relapsed following stem cell transplant.

61. The method of any one of embodiments 31-60, wherein the subject has previously received CAR-T therapy.

62. The method of embodiment 61, wherein the subject relapsed after CAR-T therapy.

63. The method of any one of embodiments 31-62, wherein the cancer is recurrent cancer.

64. The method of any one of embodiments 31-54, wherein the subject has not previously been treated for the cancer.

65. The method of any one of embodiments 31-64, wherein the subject has not been previously treated with an antibody-drug conjugate that binds to CD30.

66. The method of any one of embodiments 31-65, wherein at least 1% of the cancer cells in the subject express CD30.

67. The method of any one of embodiments 48-65, wherein the cancer is a CD30− cancer.

68. The method of any one of embodiments 31-67, wherein administering the antibody-drug conjugate to the subject results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the antibody-drug conjugate to the subject.

69. The method of any one of embodiments 31-68, wherein one or more therapeutic effects in the subject is improved after administration of the antibody-drug conjugate relative to a baseline.

70. The method of embodiment 69, wherein the one or more therapeutic effects is selected from the group consisting of: objective response rate, duration of response, time to response, progression free survival and overall survival.

71. The method of any one of embodiments 31-70, wherein the objective response rate is at least about 20%, at least about 25%, at least about 30%, at least about 35%, at least about 40%, at least about 45%, at least about 50%, at least about 60%, at least about 70%, or at least about 80%.

72. The method of any one of embodiments 31-71, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

73. The method of any one of embodiments 31-72, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

74. The method of any one of embodiments 31-73, wherein the duration of response to the conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

75. The method of any one of embodiments 1-74, wherein the subject is a human.

76. A pharmaceutical composition an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, wherein the composition is for use in the method of any one of embodiments 1-75.

77. A kit comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, and instructions for using the kit in the method of any one of embodiments 1-75.

### EXAMPLES

### Example 1: Anti-CD30 Antibody-Drug Conjugate Depleted CD30+T Regulatory Cells In Vitro

The ability of the anti-CD30 antibody-drug conjugate SGN-CD30C to deplete CD30+T regulatory (Tregs) was assessed in vitro. Inducible T regulatory cells (iTregs) were differentiated from cryopreserved CD4⁺ T cells of two normal donors (Cellero, Bothell, Washington, Cat #1023) in X-Vivo 15 media (Lonza, Cat #04-418Q) supplemented with 50 ng/ml TGFβ1 (R&D, cat #100-B), 50 ng/ml IL-2 (R&D, Cat #202-IL/CF) and a 1:100 dilution of Lipid Mixture 1 (Sigma, Cat #L0288-100 ml) with CD3/CD28 beads (Miltenyi, Cat #130-095-345) at a 1:20 bead:cell ratio for 6 days at 37° C. and 5% CO₂, followed by 2 days resting in X-Vivo 15 media only. Differentiated iTregs were then immunophenotyped prior to in vitro cytotoxicity assays with a viability dye and fluorescently labeled anti-human CD3, CD4, CD8, CD25 and Foxp3 (Biolegend and BD Pharmingen), and analyzed on an Attune NxT Flow Cytometer (Life Technologies). Following differentiation, the iTreg populations were 38% and 53% CD25⁺FoxP3⁺. Inducible Tregs from 3 additional normal donors were also ordered directly from Cellero (Cat #1042).

Cryopreserved inducible Tregs from 5 normal donors and CD4⁺CD127^{low}CD25⁺ peripheral blood Tregs from 2 normal donors (Cellero and Stem Cell Technologies) were washed and resuspended in RPMI 1640 media containing 10% FBS and put into culture with CD3/CD28 beads at a 1:4 bead:cell ratio and 10 ng/ml IL-2 for 4-5 days in a 96-well round bottom plate at 37° C. and 5% CO₂ in a titration of cAC10-7782 or non-specific control hIgG-7782. At the end of the incubation, cells were washed and stained with a Zombie Aqua viability dye and CD30 PE (Biolegend), and both total and CD30⁺ live cell counts were determined using an Attune Nxt Flow cytometer. Untreated inducible Tregs were 37-80% CD30⁺ and peripheral Tregs were 33-68% CD30⁺ at the conclusion of the assay.

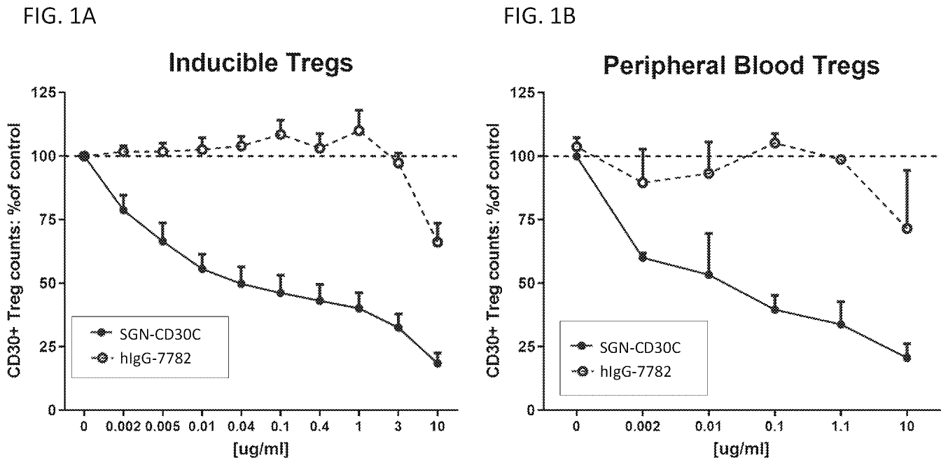

SGN-CD30C drove a dose-dependent reduction of total CD30⁺ iTreg (FIG. 1A) and peripheral blood CD30+ Tregs (FIG. 1B) compared to a non-binding control antibody-drug conjugate. Cell counts are shown as the percent of untreated control.

We claim:

1. A method of decreasing the number of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof.

2. The method of claim 1, wherein the number of CD30⁺ Treg cells is decreased relative to the number of CD30 Treg cells in the subject prior to administration of the antibody-drug conjugate.

3. A method of decreasing the activity of CD30⁺ T regulatory (Treg) cells in a subject comprising administering to the subject an antibody-drug conjugate, wherein the antibody-drug conjugate comprises an anti-CD30 antibody or an antigen-binding fragment thereof conjugated a camptothecin or a functional analog thereof or a functional derivative thereof.

4. The method of claim 3, wherein the decrease in the activity of CD30 Treg cells is relative to the activity of CD30 Treg cells in the subject prior to administration of the antibody-drug conjugate.

5. The method of claim 2, wherein the CD30+ Treg cells are inducible T regulatory (iTreg) cells.

6. The method of claim 2, wherein the CD30+ Treg cells are peripheral blood T regulatory (pbTreg) cells.

7. The method of claim 2, wherein the anti-CD30 antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

8. The method of claim 7, wherein the anti-CD30 antibody or antigen-binding fragment thereof of the antibody-drug conjugate comprises a heavy chain variable region comprising an amino acid sequence at least 85% identical to the amino acid sequence of SEQ ID NO: 7 and a light chain variable region comprising an amino acid sequence at least 85% identical to the amino acid sequence of SEQ ID NO: 8.

9. The method of claim 7, wherein the anti-CD30 antibody or antigen-binding fragment thereof of the antibody-drug conjugate comprises a heavy chain variable region comprising an amino acid sequence at least 90% identical to the amino acid sequence of SEQ ID NO: 7 and a light chain variable region comprising an amino acid sequence at least 90% identical to the amino acid sequence of SEQ ID NO: 8.

10. The method of claim 7, wherein the anti-CD30 antibody or antigen-binding fragment thereof of the antibody-drug conjugate comprises a heavy chain variable region comprising an amino acid sequence at least 95% identical to the amino acid sequence of SEQ ID NO: 7 and a light chain variable region comprising an amino acid sequence at least 95% identical to the amino acid sequence of SEQ ID NO: 8.

11. The method of claim 2, wherein the anti-CD30 antibody or antigen-binding fragment thereof of the antibody-drug conjugate comprises a heavy chain variable region comprising the amino acid sequence of SEQ ID NO: 7 and a light chain variable region comprising the amino acid sequence of SEQ ID NO: 8.

12. The method of claim 2, wherein the anti-CD30 antibody or antigen-binding fragment thereof of the antibody-drug conjugate is cAC10.

13. The method of claim 2, wherein the antibody-drug conjugate comprises an anti-CD30 antibody or an antigen-binding fragment thereof conjugated to a camptothecin or a functional analog thereof or a functional derivative thereof forming a camptothecin conjugate of Formula (IC): 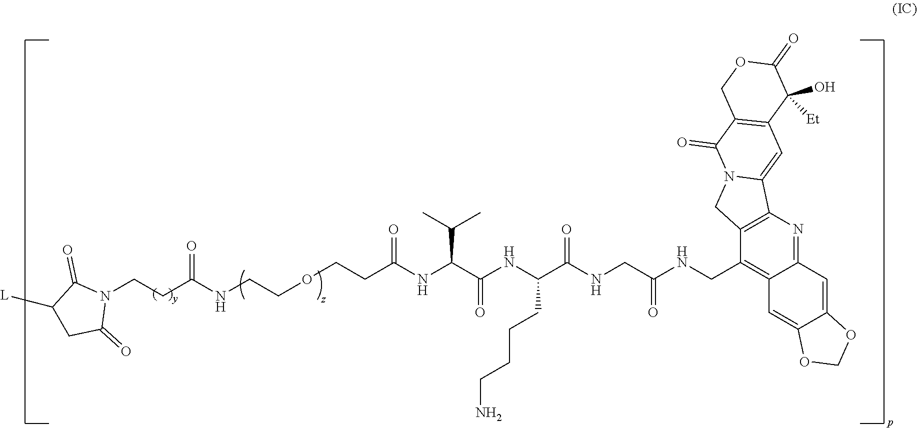
or a pharmaceutically acceptable salt thereof, wherein:
L is the anti-CD30 antibody or an antigen-binding fragment thereof,
y is 1, 2, 3, or 4, or is 1 or 4,
z is an integer from 2 to 12, or is 2, 4, 8, or 12, and
p is 1-16, or is 2, 3, 4, 5, 6, 7, 8, 9, or 10, or is 2, 4 or 8.

14. The method of claim 13, wherein y is 1.

15. The method of claim 14, wherein z is 8.

16. The method of claim 15, wherein p is 8.

17. The method of claim 16, wherein the antibody-drug conjugate is SGN-CD30C.

18. The method of claim 2, wherein the antibody-drug conjugate is administered at a dose of 0.01 mg/kg to 5 mg/kg of the subject's bodyweight.

19. The method of claim 18, wherein the antibody-drug conjugate is administered at a dose of 0.1 mg/kg to 2 mg/kg of the subject's bodyweight.

20. The method of claim 18, wherein the antibody-drug conjugate is administered at a dose of about 0.1 mg/kg of the subject's bodyweight.

21. The method of claim 18, wherein the antibody-drug conjugate is administered at a dose of about 0.5 mg/kg of the subject's bodyweight.

22. The method of claim 2, wherein the antibody-drug conjugate is administered to the subject once about every 3 weeks.

23. The method of claim 2, wherein the antibody-drug conjugate is administered to the subject once every 3 weeks.

24. The method of claim 22, wherein the antibody-drug conjugate is administered to the subject on about day 1 of about a 21-day treatment cycle.

25. The method of claim 22, wherein the antibody-drug conjugate is administered to the subject on day 1 of a 21-day treatment cycle.

26. The method of claim 2, wherein the antibody-drug conjugate is administered by intravenous infusion.

27. The method of claim 2, further comprising the administration of granulocyte-colony stimulating factor (G-CSF) to the subject.

28. The method of claim 27, wherein the G-CSF is administered 1 to 3 days after the administration of the antibody-drug conjugate.

29. The method of claim 27, wherein the G-CSF is selected from the group consisting of filgrastim, PEG-filgrastim, lenograstim, and tbo-filgrastim.

30. The method of claim 2, wherein the method further comprises administering one or more additional therapeutic agents capable of modulating the immune response.

31. The method of claim 2, wherein the subject has cancer.

32. The method of claim 31, wherein the cancer is a hematologic cancer.

33. The method of claim 31, wherein the cancer is selected from the group consisting of Hodgkin lymphoma, non-Hodgkin lymphoma, anaplastic large cell lymphoma, peripheral T-cell lymphoma, or mycosis fungoides.

34. The method of claim 33, wherein the cancer is Hodgkin lymphoma.

35. The method of claim 34, wherein the Hodgkin lymphoma is classical Hodgkin lymphoma (cHL).

36. The method of claim 33, wherein the cancer is non-Hodgkin lymphoma.

37. The method of claim 36, wherein the non-Hodgkin lymphoma is diffuse large B-cell lymphoma (DLBCL).

38. The method of claim 37, wherein the DLBCL is germinal-center B-cell like (GCB).

39. The method of claim 37, wherein the DLBCL is non-GCB.

40. The method of claim 33, wherein the cancer is anaplastic large cell lymphoma.

41. The method of claim 40, wherein the anaplastic large cell lymphoma is systemic anaplastic large cell lymphoma.

42. The method of claim 40, wherein the anaplastic large cell lymphoma is primary cutaneous anaplastic large cell lymphoma.

43. The method of claim 36, wherein the non-Hodgkin lymphoma is a mature T-cell lymphoma.

44. The method of claim 36, wherein the non-Hodgkin lymphoma is cutaneous T-cell lymphoma (CTCL).

45. The method of claim 33, wherein the cancer is peripheral T-cell lymphoma.

46. The method of claim 45, wherein the peripheral T-cell lymphoma is angioimmunoblastic T-cell lymphoma.

47. The method of claim 33, wherein the cancer is mycosis fungoides.

48. The method of claim 31, wherein the cancer is a non-hematologic cancer.

49. The method of claim 48, wherein the non-hematologic cancer is a carcinoma.

50. The method of claim 48, wherein the non-hematologic cancer is a sarcoma.

51. The method of claim 48, wherein the non-hematologic cancer is a solid tumor.

52. The method of claim 31, wherein the cancer is an advanced stage cancer.

53. The method of claim 52, wherein the advanced stage cancer is a stage 3 or stage 4 cancer.

54. The method of claim 52, wherein the advanced stage cancer is metastatic cancer.

55. The method of claim 31, wherein the subject has been previously treated with one or more therapeutic agents and did not respond to the treatment.

56. The method of claim 31, wherein the subject has been previously treated with one or more therapeutic agents and relapsed after the treatment.

57. The method of claim 31, wherein the subject has been previously treated with one or more therapeutic agents and has experienced disease progression during treatment.

58. The method of claim 31, wherein the subject has previously received allogenic stem cell transplant to treat the cancer.

59. The method of claim 31, wherein the subject has previously received autologous stem cell transplant to treat the cancer.

60. The method of claim 58, wherein the subject relapsed following stem cell transplant.

61. The method of claim 31, wherein the subject has previously received CAR-T therapy.

62. The method of claim 61, wherein the subject relapsed after CAR-T therapy.

63. The method of claim 31, wherein the cancer is recurrent cancer.

64. The method of claim 31, wherein the subject has not previously been treated for the cancer.

65. The method of claim 31, wherein the subject has not been previously treated with an antibody-drug conjugate that binds to CD30.

66. The method of claim 31, wherein at least 1% of the cancer cells in the subject express CD30.

67. The method of claim 31, wherein the cancer is a CD30− cancer.

68. The method of claim 31, wherein administering the antibody-drug conjugate to the subject results in a depletion of cancer cells by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or about 100% compared to the amount of cancer cells before administering the antibody-drug conjugate to the subject.

69. The method of claim 31, wherein one or more therapeutic effects in the subject is improved after administration of the antibody-drug conjugate relative to a baseline.

70. The method of claim 69, wherein the one or more therapeutic effects is selected from the group consisting of: objective response rate, duration of response, time to response, progression free survival and overall survival.

71. The method of claim 65, wherein the objective response rate is at least about 20%, at least about 25%, at least about 30%, at least about 35%, at least about 40%, at least about 45%, at least about 50%, at least about 60%, at least about 70%, or at least about 80%.

72. The method of claim 65, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

73. The method of claim 65, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

74. The method of claim 31, wherein the duration of response to the conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

75. The method of any one of claims 1-74, wherein the subject is a human.

76. A pharmaceutical composition an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, wherein the composition is for use in the method of claim 75.

77. A kit comprising an antibody-drug conjugate that binds to CD30, wherein the antibody-drug conjugate comprises an anti-CD30 antibody or an antigen-binding fragment thereof conjugated to a camptothecin or a functional analog thereof or a functional derivative thereof, and instructions for using the kit in the method of claim 75.


SEQUENCE LISTING (16 sequences)

SEQ ID NO: 1 - protein, 5 aa, Artificial Sequence
  Synthetic Construct
DYYIT

SEQ ID NO: 2 - protein, 17 aa, Artificial Sequence
  Synthetic Construct
WIYPGSGNTKYNEKFKG

SEQ ID NO: 3 - protein, 8 aa, Artificial Sequence
  Synthetic Construct
YGNYWFAY

SEQ ID NO: 4 - protein, 15 aa, Artificial Sequence
  Synthetic Construct
KASQSVDFDGDSYMN

SEQ ID NO: 5 - protein, 7 aa, Artificial Sequence
  Synthetic Construct
AASNLES

SEQ ID NO: 6 - protein, 9 aa, Artificial Sequence
  Synthetic Construct
QQSNEDPWT

SEQ ID NO: 7 - protein, 117 aa, Artificial Sequence
  Synthetic Construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, Artificial Sequence
  Synthetic Construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 30 aa, Artificial Sequence
  Synthetic Construct
QIQLQQSGPEVVKPGASVKISCKASGYTFT

SEQ ID NO: 10 - protein, 14 aa, Artificial Sequence
  Synthetic Construct
WVKQKPGQGLEWIG

SEQ ID NO: 11 - protein, 32 aa, Artificial Sequence
  Synthetic Construct
KATLTVDTSSSTAFMQLSSLTSEDTAVYFCAN

SEQ ID NO: 12 - protein, 11 aa, Artificial Sequence
  Synthetic Construct
WGQGTQVTVSA

SEQ ID NO: 13 - protein, 23 aa, Artificial Sequence
  Synthetic Construct
DIVLTQSPASLAVSLGQRATISC

SEQ ID NO: 14 - protein, 15 aa, Artificial Sequence
  Synthetic Construct
WYQQKPGQPPKVLIY

SEQ ID NO: 15 - protein, 32 aa, Artificial Sequence
  Synthetic Construct
GIPARFSGSGSGTDFTLNIHPVEEEDAATYYC

SEQ ID NO: 16 - protein, 10 aa, Artificial Sequence
  Synthetic Construct
FGGGTKLEIK